(19) United States
(12) Patent Application Publication
(10) Pub. No.: US 2023/0260608 A1
(43) Pub. Date: Aug. 17, 2023

(54) RELATIONSHIP PREDICTION

(71) Applicant: Ancestry.com DNA, LLC, Lehi, UT (US)

(72) Inventors: Luong Ruiz, San Bruno, CA (US); Ross Eugene Curtis, Cedar Hills, UT (US)

(21) Appl. No.: 18/101,075

(22) Filed: Jan. 24, 2023

Related U.S. Application Data
(60) Provisional application No. 63/310,815, filed on Feb. 16, 2022.

(51) Int. Cl. G16H 10/60; G06N 5/02

(52) U.S. Cl. CPC G16H 10/60; G06N 5/02

(57) ABSTRACT

Disclosed herein relates to a method that improves the prediction of relationships between individuals. Relationship prediction systems, methods, and computer-program products are described. Relationship prediction of a most recent common ancestor and a most likely relative is performed using a multilabel-multiclass classification based on k-nearest neighbors classification, logistic regression, and/or other classification approaches. Predicting a most recent common ancestor narrows the number of possible relationships between a user of a genealogical research service and a relative and facilitates more intuitive discoveries and more specific identification of a most likely relationship.

### CROSS-REFERENCE TO RELATED APPLICATION

The present application claims the benefit of U.S. Provisional Patent Application No. 63/310,815 filed on Feb. 16, 2022, which is hereby incorporated by reference in its entirety.

### FIELD

The disclosed embodiments relate to systems, methods and/or computer-program products configured for predicting or mapping relationships, such as relationships across or pertaining to generations.

### BACKGROUND

Although humans are, genetically speaking, almost entirely identical, small differences in human DNA are responsible for some observed variations between individuals. Most of the mutations that are passed down to descendants are related to single-nucleotide polymorphism (SNP). SNP is a substitution of a single nucleotide that occurs at a specific position in the genome. Learning about population structure from genetic polymorphism data is an important topic in genetics.

Identifying segments of IBD DNA between pairs of genotyped individuals is useful in many applications. Therefore, numerous methods have been developed to perform IBD analysis (Purcell et al. 2007, Gusev et al. 2009, Browning and Browning 2011, Browning and Browning 2013). However, these approaches do not scale for continuously growing, very large datasets. For example, the existing GERMLINE implementation is designed to take a single input file containing all individuals to be compared against one another. While appropriate for the case in which all samples are genotyped and analyzed simultaneously, this approach is not practical when samples are collected incrementally.

Attempts have been made to predict the exact nature of the relationship between people who are determined to be relatives. Beyond a certain threshold, however, the prediction becomes so attenuated—and so wanting for specificity—that it is both confusing and of little use to ordinary users of a genealogical research service. For example, the prediction may include a broad range of possible relationships or match categories that include terms such as “third cousin” or “second cousin thrice removed” that are not intuitive or meaningful to a user. Further, many such predictions can include four or five plausible predictions for a relative, making the relationship prediction highly tenuous and confusing. That is, genetic matches determined using IBD or other methods often have limited accuracy and utility in determining a particular relationship given the number of potential relationships at different degrees of separation between individuals. For example, a third cousin once removed, a half third cousin, a half second cousin twice removed, and a second cousin three times removed may be equally plausible relationships based on a degree of genetic similarity.

### SUMMARY

Various embodiments described herein relate to a computer-implemented method, including: receiving a first genetic dataset of a target individual; receiving a second genetic dataset of a match individual, the match individual being a genetic match of the target individual; extracting a plurality of features between the target individual and the match individual, wherein the plurality of features comprise one or more genetic features shared between the first and second genetic datasets and an age difference between the target individual and the match individual; inputting the plurality of features to a machine-learning model; and predicting, using the machine-learning model, a number of generations between a most recent common ancestor (MRCA) and the target individual and a number of generations between the MRCA and the match individual.

In some embodiments, the match individual is identified by identity by descent (IBD) segments shared between the first genetic dataset and the second genetic dataset.

In some embodiments, the match individual is identified by centimorgans (cM) shared, a number of shared segments, or other genetic similarity with the target individual.

In some embodiments, the genetic features comprise cM shared and the number of shared segments, e.g. IBD segments, between the two individuals.

In some embodiments, the predicted number of generations between the MRCA and the target individual and the number of generations between the MRCA and the match individual are used to generate predicted relationships between the target individual and the match individual.

In some embodiments, the number of generations between the MRCA and the target individual and the number of generations between the MRCA and the match individual are used in combination with cM shared between the target individual and the match individual, a number of shared DNA segments, and age difference between the target individual and match individual to generate predicted relationships for the target individual and the match individual.

In some embodiments, the machine learning model is trained on training samples, each training sample comprising an age difference between a pair of matched individuals, cM between the pair, and a number of shared segments between the pair.

In some embodiments, training of the machine learning model comprises receiving training samples that comprise age differences between pairs of matched individuals and known generation data, inputting the training samples to the machine learning model to generate predicted generations, comparing the predicted generations to known generation data in the training samples, and adjusting weights of the machine learning model based on the comparison.

In yet another embodiment, a non-transitory computer-readable medium that is configured to store instructions is described. The instructions, when executed by one or more processors, cause the one or more processors to perform a process that includes steps described in the above computer-implemented methods or described in any embodiments of this disclosure. In yet another embodiment, a system may include one or more processors and a storage medium that is configured to store instructions. The instructions, when executed by one or more processors, cause the one or more processors to perform a process that includes steps described in the above computer-implemented methods or described in any embodiments of this disclosure.

These and other features of the present disclosure will become better understood regarding the following description, appended claims, and accompanying drawings.

### BRIEF DESCRIPTION OF THE DRAWINGS

FIG. 1 illustrates a diagram of a system environment of an example computing system, in accordance with some embodiments.

FIG. 2 is a block diagram of an architecture of an example computing system, in accordance with some embodiments.

FIG. 3A is a flowchart depicting an example process for determining a number of generations between a MRCA and a target individual, and a number of generations between the MRCA and a match individual.

FIG. 3B is an example diagram of a neural network, in accordance with some embodiments.

FIG. 4A is an example family tree depicting potential relationships between a target individual and other individuals in a database, in accordance with some embodiments.

FIG. 4B is an example family tree depicting potential relationships between a target individual, MRCA, and a match individual, according to some embodiments.

FIG. 5 depicts an example method for training a machine learning model for relationship predictions.

FIG. 6A is a confusion matrix for predictions in the M3 relationship category, in accordance with some embodiments.

FIG. 6B is a confusion matrix for predictions in the M3 relationship category with normalization, in accordance with some embodiments.

FIG. 7 depicts errors for targets and matches at meiosis levels M4-M7, in accordance with some embodiments.

FIG. 8A depicts avuncular and grandparent relationship samples plotted as a function of a number of shared segments and cM shared, in accordance with some embodiments.

FIG. 8B depicts half-sibling relationships samples plotted as a function of a number of shared segments and cM shared, in accordance with some embodiments.

FIG. 8C depicts half-sibling, avuncular, and grandparent relationship samples generated using a combination of cM shared, number of shared segments, and age difference, in accordance with some embodiments.

FIG. 9 illustrates a user interface for displaying predicted relationship results, in accordance with some embodiments.

FIG. 10 illustrates a user interface for displaying a family tree, in accordance with some embodiments.

FIG. 11 is a block diagram of an example computing device, in accordance with some embodiments.

The figures depict various embodiments for purposes of illustration only. One skilled in the art will readily recognize from the following discussion that alternative embodiments of the structures and methods illustrated herein may be employed without departing from the principles described herein.

### DETAILED DESCRIPTION

The figures (FIGs.) and the following description relate to preferred embodiments by way of illustration only. One of skill in the art may recognize alternative embodiments of the structures and methods disclosed herein as viable alternatives that may be employed without departing from the principles of what is disclosed.

Reference will now be made in detail to several embodiments, examples of which are illustrated in the accompanying figures. It is noted that wherever practicable similar or like reference numbers may be used in the figures and may indicate similar or like functionality. The figures depict embodiments of the disclosed system (or method) for purposes of illustration only. One skilled in the art will readily recognize from the following description that alternative embodiments of the structures and methods illustrated herein may be employed without departing from the principles described herein.

A better understanding of different embodiments of the disclosure may be had from the following description read with the accompanying drawings in which like reference characters refer to like elements. While the disclosure is susceptible to various modifications and alternative constructions, certain illustrative embodiments are in the drawings and are described below. It should be understood, however, there is no intention to limit the disclosure to the embodiments disclosed, but on the contrary, the intention covers all modifications, alternative constructions, combinations, and equivalents falling within the spirit and scope of the disclosure. Unless a term is defined in this disclosure to possess a described meaning, there is no intent to limit the meaning of such term, either expressly or indirectly, beyond its plain or ordinary meaning.

Reference characters are provided in the claims for explanatory purposes only and are not intended to limit the scope of the claims or restrict each claim limitation to the element in the drawings and identified by the reference character.

For ease of understanding the disclosed embodiments of systems and methods for relationship prediction, certain modules and features are described independently. The modules and features may be synergistically combined in some embodiments to provide a relationship prediction system, method, and/or computer-program product.

Reference will now be made in detail to several embodiments, examples of which are illustrated in the accompanying figures. The figures depict embodiments of the disclosed relationship-prediction systems (or methods or computer-program products) for purposes of illustration only. One skilled in the art will readily recognize from the following description that alternative embodiments of the structures and methods illustrated herein may be employed without departing from the principles described herein.

### Configuration Overview

Relationship prediction embodiments advantageously address the problem of existing genealogical and DNA research services being ill-suited to predicting relationships in a way that is meaningful and intuitive to users thereof. In some embodiments, relationship prediction systems, methods, and computer-program products are configured to reduce a number of possible relationships between a user and a match individual as well as to predict a most recent common ancestor (“MRCA”) through whom the user, which may be referred to as a target individual, and the match individual are connected.

It has been found that users respond more favorably to a relationship prediction when the relationship prediction includes a MRCA, as terms such as “great-grandmother,” “great-great-grandfather,” etc. are more intuitively meaningful to users and make an associated prediction, such as “second cousin,” who may be related to the user, or target individual, via a great grandparent, more understandable and relatable. Providing a predicted MRCA in addition to a predicted relationship between the user and their relative renders the prediction clear and specific where existing methods are vague and confusing, as most users do not have an intuitive sense for how they are related to their second cousin or first cousin twice removed, but they are more likely to intuitively understand a common MRCA relationship such as “great-grandfather.” In some embodiments, a most-likely MRCA and corresponding most-likely relationship prediction are presented to a user. A second-most likely MRCA and corresponding second-most likely relationship prediction are also presented in embodiments, and so on.

Accurate prediction of a MRCA, however, is not a trivial task, and the difficulty of the prediction increases exponentially as the relationship between the user and their relative becomes more attenuated. It has been found that a multilabel-multiclass classification approach, utilizing features such as cM shared, number of shared segments such as IBD segments, and/or age difference between the user and their relative, may be used to predict a number of generations between the MRCA and the user and their relative. This prediction advantageously facilitates a prediction regarding a most likely MRCA and consequently a most likely relationship between the user and their relative. As used herein, “user” may be described or used interchangeably with “target,” “target individual,” or “target person,” and “relative” may be described or used interchangeably with “match,” “match individual,” or “match person.”

The multilabel-multiclass classification may be performed using a k-nearest neighbors approach. In other embodiments, the multiclass-multilabel classification may be performed using one or more of a decision tree classifier approach, an extra tree classifier approach, an extremely randomized trees classifier (which may be referred to as an extra trees classifier) approach, a radius neighbors classifier approach, a random forest classifier approach, modifications and/or combinations thereof, or any other suitable approach.

Different approaches may be used for different relationships, for example a different classification approach may be used for parent-child relationships, for grandparent/avuncular/half-sibling relationships, and so on.

The relationship prediction embodiments may be configured to facilitate prediction of a MRCA and one or more most likely relationships for a plurality of different relationship tiers. In some embodiments, and as seen below in Table 1, a “one-meiosis-event relationship” or “M1 relationship” corresponds to a parent-child relationship, a “two-meiosis-event relationship” or “M2 relationship” corresponds to a full sibling relationship, a “three-meiosis-event relationship” or “M3 relationship” corresponds to half-sibling, grandparent-grandchild, or avuncular relationship, a “four-meiosis-event relationship” or “M4 relationship” corresponds to a first cousin, great grandparent to grandchild, half avuncular, or great avuncular relationship, a “five-meiosis-event relationship” or “M5 relationship” corresponds to a first cousin once removed, half first cousin, or half great avuncular relationship, a “six-meiosis-event relationship” or “M6 relationship” corresponds to a second cousin, first cousin twice removed, or half first cousin once removed relationship, a “seven-meiosis-event relationship” or “M7 relationship” corresponds to a second cousin once removed, half second cousin, first cousin thrice removed, or half first cousin once removed relationship, an “eight-meiosis-event relationship” or “M8 relationship” corresponds to a third cousin, or a second cousin twice removed relationship, and a “nine-meiosis-event relationship” or “M9 relationship” corresponds to a third cousin once removed, or second cousin thrice removed relationship, and so on.

TABLE 1

| Number of | | Possible |
|---|---|---|
| Meiosis Events | Abbreviation | Relationships |
| One-meiosis-event | “M1” | Parent-child |
| relationship | | |
| Two-meiosis-event | “M2” | Full Siblings |
| relationship | | |
| Three-meiosis-event | “M3” | Half-sibling, Grandparent, |
| relationship | | Avuncular |
| Four-meiosis-event | “M4” | First cousin, |
| relationship | | Great grandparent, |
| | | Half avuncular |
| Five-meiosis-event | “M5” | First cousin once removed, |
| relationship | | Half first cousin, |
| | | Great-great avuncular, |
| | | Great-great grandparent |
| Six-meiosis-event | “M6” | Second cousin, |
| relationship | | First cousin twice removed, |
| | | Half first cousin once removed, |
| | | Half two-generation avuncular, |
| | | Three-generation grandparent |
| Seven-meiosis-event | “M7” | Second cousin once removed, |
| relationship | | Half second cousin, |
| | | First cousin thrice removed, |
| | | Half first cousin twice removed, |
| | | Four-generation avuncular, |
| | | Half three-generation avuncular, |
| | | Four-generation grandparent |
| Eight-meiosis-event | “M8” | Third cousin, |
| relationship | | Second cousin twice removed, |
| | | Half second cousin once removed, |
| | | First cousin four times removed, |
| | | Half first cousin three times removed, |
| | | Five-generation avuncular, |
| | | Half four-generation avuncular, |
| | | Five-generation grandparent |
| Nine-meiosis-event | “M9” | Third cousin once removed, |
| relationship | | Half third cousin, |
| | | Second cousin thrice removed, |
| | | Half second cousin twice removed, |
| | | First cousin five times removed, |
| | | Half first cousin four times removed, |
| | | Six-generation avuncular, |
| | | Half five-generation avuncular |
| Ten-meiosis-event | “M10” | Fourth cousin, |
| relationship | | Third cousin twice removed, |
| | | Half third cousin once removed, |
| | | Second cousin four times removed, |
| | | Half second cousin thrice removed, |
| | | First cousin six times removed, |
| | | Half first cousin five times removed, |
| | | Seven-generation avuncular, |
| | | Half six-generation avuncular |

In some embodiments, M3 relationship predictions (e.g., those pertaining to grandparent/avuncular/half-sibling relationships) are predicted using a logistic regression approach. M4-M7 relationships, by contrast, are predicted using a k-nearest neighbors approach. While logistic regression for M3 relationship predictions and k-nearest neighbors for M4-M7 relationships are described, it will be appreciated that the disclosure is by no means limited thereto. Rather, any suitable approach or combination of approaches may be used for any suitable level of relationship. For example, both logistic regression and k-nearest neighbor may be performed in parallel for M3-M7 relationship predictions, with a suitable prediction selected therebetween.

Features for performing relationship predictions may be drawn from user data, e.g., based on the cM, number of segments shared, age differences between target and match, etc. One or more machine-learned models for performing the prediction may be trained using data obtained from, e.g., a stitched genealogical tree database. The stitched genealogical tree database may comprise one or more distinct databases comprising, e.g., a genealogical tree database and a stitched tree database comprising a stitched tree formed from stitched-together genealogical trees. In the stitched tree, entity resolution is and/or has been performed to cluster together instances of the same individual occurring in separate trees.

Thus details entered separately (e.g., in separate trees, by different contributors) about a same individual can be jointly accessed, yielding more-efficient and automated access to a greater quantity of information about a greater number of individuals. The stitched genealogical tree database may be provided, maintained, and/or utilized as described in, e.g., U.S. Patent Application Publication No. 2020/0394188, published Dec. 17, 2020, U.S. Pat. No. 11,347,798, granted May 31, 2022, U.S. Patent Application Publication No. 2021/0319003, published Oct. 14, 2021, U.S. Pat. No. 11,321,361, granted May 3, 2022, each of which is hereby incorporated in its entirety by reference.

Training data for the k-nearest neighbors approach or model may include labels from approximately 35,000 matched pairs, including associated DNA results and genealogical tree information. The matched pairs were identified from a stitched genealogical tree database as described above. The use of a large number of matched pairs from a stitched genealogical tree database advantageously allowed the approach to overwhelm errors in the predictions with correct information, thereby arriving ultimately at accurate predictions.

These and other features of the present disclosure will become better understood regarding the following description, appended claims, and accompanying drawings.

### Example System Environment

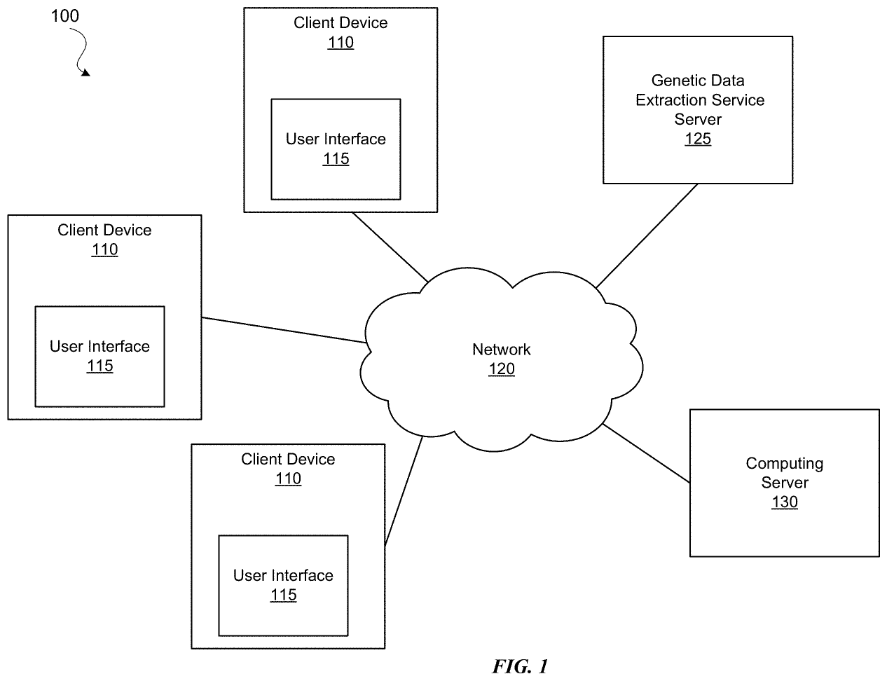

FIG. 1 illustrates a diagram of a system environment 100 of an example computing server 130, in accordance with some embodiments. The system environment 100 shown in FIG. 1 includes one or more client devices 110, a network 120, a genetic data extraction service server 125, and a computing server 130. In various embodiments, the system environment 100 may include fewer or additional components. The system environment 100 may also include different components.

The client devices 110 are one or more computing devices capable of receiving user input as well as transmitting and/or receiving data via a network 120. Example computing devices include desktop computers, laptop computers, personal digital assistants (PDAs), smartphones, tablets, wearable electronic devices (e.g., smartwatches), smart household appliances (e.g., smart televisions, smart speakers, smart home hubs), Internet of Things (IoT) devices or other suitable electronic devices. A client device 110 communicates to other components via the network 120. Users may be customers of the computing server 130 or any individuals who access the system of the computing server 130, such as an online website or a mobile application. In some embodiments, a client device 110 executes an application that launches a graphical user interface (GUI) for a user of the client device 110 to interact with the computing server 130. The GUI may be an example of a user interface 115. A client device 110 may also execute a web browser application to enable interactions between the client device 110 and the computing server 130 via the network 120. In another embodiment, the user interface 115 may take the form of a software application published by the computing server 130 and installed on the user device 110. In yet another embodiment, a client device 110 interacts with the computing server 130 through an application programming interface (API) running on a native operating system of the client device 110, such as IOS or ANDROID.

The network 120 provides connections to the components of the system environment 100 through one or more sub-networks, which may include any combination of local area and/or wide area networks, using both wired and/or wireless communication systems. In some embodiments, a network 120 uses standard communications technologies and/or protocols. For example, a network 120 may include communication links using technologies such as Ethernet, 802.11, worldwide interoperability for microwave access (WiMAX), 3G, 4G, Long Term Evolution (LTE), 5G, code division multiple access (CDMA), digital subscriber line (DSL), etc. Examples of network protocols used for communicating via the network 120 include multiprotocol label switching (MPLS), transmission control protocol/Internet protocol (TCP/IP), hypertext transport protocol (HTTP), simple mail transfer protocol (SMTP), and file transfer protocol (FTP). Data exchanged over a network 120 may be represented using any suitable format, such as hypertext markup language (HTML) or extensible markup language (XML). In some embodiments, all or some of the communication links of a network 120 may be encrypted using any suitable technique or techniques such as secure sockets layer (SSL), transport layer security (TLS), virtual private networks (VPNs), Internet Protocol security (IPsec), etc. The network 120 also includes links and packet switching networks such as the Internet.

Individuals, who may be customers of a company operating the computing server 130, provide biological samples for analysis of their genetic data. Individuals may also be referred to as users. In some embodiments, an individual uses a sample collection kit to provide a biological sample (e.g., saliva, blood, hair, tissue) from which genetic data is extracted and determined according to nucleotide processing techniques such as amplification and sequencing. Amplification may include using polymerase chain reaction (PCR) to amplify segments of nucleotide samples. Sequencing may include sequencing of deoxyribonucleic acid (DNA) sequencing, ribonucleic acid (RNA) sequencing, etc. Suitable sequencing techniques may include Sanger sequencing and massively parallel sequencing such as various next-generation sequencing (NGS) techniques including whole genome sequencing, pyrosequencing, sequencing by synthesis, sequencing by ligation, and ion semiconductor sequencing. In some embodiments, a set of SNPs (e.g., 300,000) that are shared between different array platforms (e.g., Illumina OmniExpress Platform and Illumina HumanHap 650Y Platform) may be obtained as genetic data. Genetic data extraction service server 125 receives biological samples from users of the computing server 130. The genetic data extraction service server 125 performs sequencing of the biological samples and determines the base pair sequences of the individuals. The genetic data extraction service server 125 generates the genetic data of the individuals based on the sequencing results. The genetic data may include data sequenced from DNA or RNA and may include base pairs from coding and/or noncoding regions of DNA.

The genetic data may take different forms and include information regarding various biomarkers of an individual. For example, in some embodiments, the genetic data may be the base pair sequence of an individual. The base pair sequence may include the whole genome or a part of the genome such as certain genetic loci of interest. In another embodiment, the genetic data extraction service server 125 may determine genotypes from sequencing results, for example by identifying genotype values of single nucleotide polymorphisms (SNPs) present within the DNA. The results in this example may include a sequence of genotypes corresponding to various SNP sites. A SNP site may also be referred to as a SNP loci. A genetic locus is a segment of a genetic sequence. A locus can be a single site or a longer stretch. The segment can be a single base long or multiple bases long. In some embodiments, the genetic data extraction service server 125 may perform data pre-processing of the genetic data to convert raw sequences of base pairs to sequences of genotypes at target SNP sites. Since a typical human genome may differ from a reference human genome at only several million SNP sites (as opposed to billions of base pairs in the whole genome), the genetic data extraction service server 125 may extract only the genotypes at a set of target SNP sites and transmit the extracted data to the computing server 130 as the genetic dataset of an individual. SNPs, base pair sequence, genotype, haplotype, RNA sequences, protein sequences, and phenotypes are examples of biomarkers.

The computing server 130 performs various analyses of the genetic data, genealogy data, and users' survey responses to generate results regarding the phenotypes and genealogy of users of computing server 130. Depending on the embodiments, the computing server 130 may also be referred to as an online server, a personal genetic service server, a genealogy server, a family tree building server, and/or a social networking system. The computing server 130 receives genetic data from the genetic data extraction service server 125 and stores the genetic data in the data store of the computing server 130. The computing server 130 may analyze the data to generate results regarding the genetics or genealogy of users. The results regarding the genetics or genealogy of users may include the ethnicity compositions of users, paternal and maternal genetic analysis, identification or suggestion of potential family relatives, ancestor information, analyses of DNA data, potential or identified traits such as phenotypes of users (e.g., diseases, appearance traits, other genetic characteristics, and other non-genetic characteristics including social characteristics), etc. The computing server 130 may present or cause the user interface 115 to present the results to the users through a GUI displayed at the client device 110. The results may include graphical elements, textual information, data, charts, and other elements such as family trees.

In some embodiments, the computing server 130 also allows various users to create one or more genealogical profiles of the user. The genealogical profile may include a list of individuals (e.g., ancestors, relatives, friends, and other people of interest) who are added or selected by the user or suggested by the computing server 130 based on the genealogical records and/or genetic records. The user interface 115 controlled by or in communication with the computing server 130 may display the individuals in a list or as a family tree such as in the form of a pedigree chart. In some embodiments, subject to user's privacy setting and authorization, the computing server 130 may allow information generated from the user's genetic dataset to be linked to the user profile and to one or more of the family trees. The users may also authorize the computing server 130 to analyze their genetic dataset and allow their profiles to be discovered by other users.

### Example Computing Server Architecture

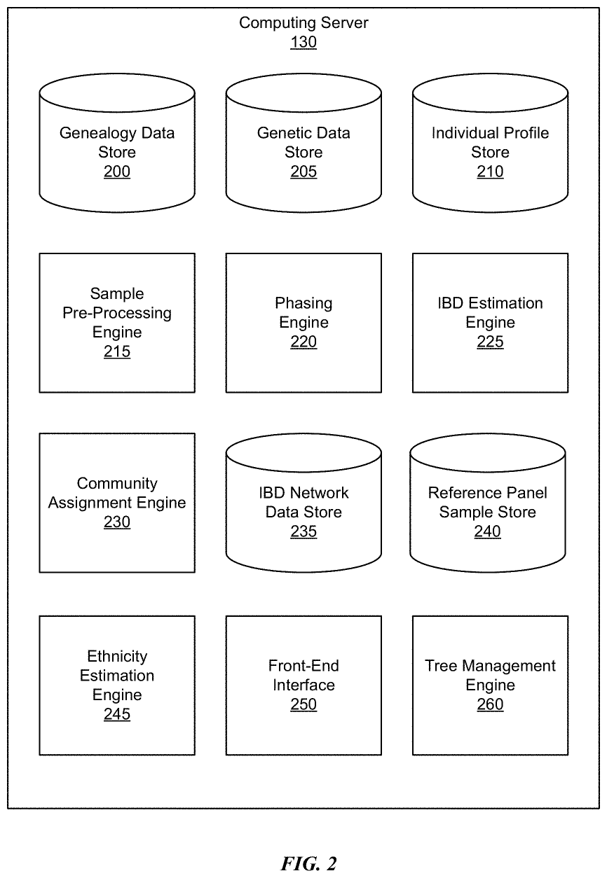

FIG. 2 is a block diagram of an architecture of an example computing server 130, in accordance with some embodiments. In the embodiment shown in FIG. 2, the computing server 130 includes a genealogy data store 200, a genetic data store 205, an individual profile store 210, a sample pre-processing engine 215, a phasing engine 220, an identity by descent (IBD) estimation engine 225, a community assignment engine 230, an IBD network data store 235, a reference panel sample store 240, an ethnicity estimation engine 245, a front-end interface 250, and a tree management engine 260. The functions of the computing server 130 may be distributed among the elements in a different manner than described. In various embodiments, the computing server 130 may include different components and fewer or additional components. Each of the various data stores may be a single storage device, a server controlling multiple storage devices, or a distributed network that is accessible through multiple nodes (e.g., a cloud storage system).

The computing server 130 stores various data of different individuals, including genetic data, genealogy data, and survey response data. The computing server 130 processes the genetic data of users to identify shared identity-by-descent (IBD) segments between individuals. The genealogy data and survey response data may be part of user profile data. The amount and type of user profile data stored for each user may vary based on the information of a user, which is provided by the user as she creates an account and profile at a system operated by the computing server 130 and continues to build her profile, family tree, and social network at the system and to link her profile with her genetic data. Users may provide data via the user interface 115 of a client device 110. Initially and as a user continues to build her genealogical profile, the user may be prompted to answer questions related to the basic information of the user (e.g., name, date of birth, birthplace, etc.) and later on more advanced questions that may be useful for obtaining additional genealogy data. The computing server 130 may also include survey questions regarding various traits of the users such as the users' phenotypes, characteristics, preferences, habits, lifestyle, environment, etc.

Genealogy data may be stored in the genealogy data store 200 and may include various types of data that are related to tracing family relatives of users. Examples of genealogy data include names (first, last, middle, suffixes), gender, birth locations, date of birth, date of death, marriage information, spouse's information kinships, family history, dates and places for life events (e.g., birth and death), other vital data, and the like. In some instances, family history can take the form of a pedigree of an individual (e.g., the recorded relationships in the family). The family tree information associated with an individual may include one or more specified nodes. Each node in the family tree represents the individual, an ancestor of the individual who might have passed down genetic material to the individual, and the individual's other relatives including siblings, cousins, and offspring in some cases. Genealogy data may also include connections and relationships among users of the computing server 130. The information related to the connections among a user and her relatives that may be associated with a family tree may also be referred to as pedigree data or family tree data.

In addition to user-input data, genealogy data may also take other forms that are obtained from various sources such as public records and third-party data collectors. For example, genealogical records from public sources include birth records, marriage records, death records, census records, court records, probate records, adoption records, obituary records, etc. Likewise, genealogy data may include data from one or more family trees of an individual, the Ancestry World Tree system, a Social Security Death Index database, the World Family Tree system, a birth certificate database, a death certificate database, a marriage certificate database, an adoption database, a draft registration database, a veterans database, a military database, a property records database, a census database, a voter registration database, a phone database, an address database, a newspaper database, an immigration database, a family history records database, a local history records database, a business registration database, a motor vehicle database, and the like.

Furthermore, the genealogy data store 200 may also include relationship information inferred from the genetic data stored in the genetic data store 205 and information received from the individuals. For example, the relationship information may indicate which individuals are genetically related, how they are related, how many generations back they share common ancestors, lengths and locations of IBD segments shared, which genetic communities an individual is a part of, variants carried by the individual, and the like.

The computing server 130 maintains genetic datasets of individuals in the genetic data store 205. A genetic dataset of an individual may be a digital dataset of nucleotide data (e.g., SNP data) and corresponding metadata. A genetic dataset may contain data on the whole or portions of an individual's genome. The genetic data store 205 may store a pointer to a location associated with the genealogy data store 200 associated with the individual. A genetic dataset may take different forms. In some embodiments, a genetic dataset may take the form of a base pair sequence of the sequencing result of an individual. A base pair sequence dataset may include the whole genome of the individual (e.g., obtained from a whole-genome sequencing) or some parts of the genome (e.g., genetic loci of interest).

In another embodiment, a genetic dataset may take the form of sequences of genetic markers. Examples of genetic markers may include target SNP loci (e.g., allele sites) filtered from the sequencing results. A SNP locus that is single base pair long may also be referred to a SNP site. A SNP locus may be associated with a unique identifier. The genetic dataset may be in a form of diploid data that includes a sequencing of genotypes, such as genotypes at the target SNP loci, or the whole base pair sequence that includes genotypes at known SNP loci and other base pair sites that are not commonly associated with known SNPs. The diploid dataset may be referred to as a genotype dataset or a genotype sequence. Genotype may have a different meaning in various contexts. In one context, an individual's genotype may refer to a collection of diploid alleles of an individual. In other contexts, a genotype may be a pair of alleles present on two chromosomes for an individual at a given genetic marker such as a SNP site.

Genotype data for a SNP site may include a pair of alleles. The pair of alleles may be homozygous (e.g., A-A or G-G) or heterozygous (e.g., A-T, C-T). Instead of storing the actual nucleotides, the genetic data store 205 may store genetic data that are converted to bits. For a given SNP site, oftentimes only two nucleotide alleles (instead of all 4) are observed. As such, a 2-bit number may represent a SNP site. For example, 00 may represent homozygous first alleles, 11 may represent homozygous second alleles, and 01 or 10 may represent heterozygous alleles. A separate library may store what nucleotide corresponds to the first allele and what nucleotide corresponds to the second allele at a given SNP site.

A diploid dataset may also be phased into two sets of haploid data, one corresponding to a first parent side and another corresponding to a second parent side. The phased datasets may be referred to as haplotype datasets or haplotype sequences. Similar to genotype, haplotype may have a different meaning in various contexts. In one context, a haplotype may also refer to a collection of alleles that corresponds to a genetic segment. In other contexts, a haplotype may refer to a specific allele at a SNP site. For example, a sequence of haplotypes may refer to a sequence of alleles of an individual that are inherited from a parent.

The individual profile store 210 stores profiles and related metadata associated with various individuals appeared in the computing server 130. A computing server 130 may use unique individual identifiers to identify various users and other non-users that might appear in other data sources such as ancestors or historical persons who appear in any family tree or genealogy database. A unique individual identifier may be a hash of certain identification information of an individual, such as a user's account name, user's name, date of birth, location of birth, or any suitable combination of the information. The profile data related to an individual may be stored as metadata associated with an individual's profile. For example, the unique individual identifier and the metadata may be stored as a key-value pair using the unique individual identifier as a key.

An individual's profile data may include various kinds of information related to the individual. The metadata about the individual may include one or more pointers associating genetic datasets such as genotype and phased haplotype data of the individual that are saved in the genetic data store 205. The metadata about the individual may also be individual information related to family trees and pedigree datasets that include the individual. The profile data may further include declarative information about the user that was authorized by the user to be shared and may also include information inferred by the computing server 130. Other examples of information stored in a user profile may include biographic, demographic, and other types of descriptive information such as work experience, educational history, gender, hobbies, or preferences, location and the like. In some embodiments, the user profile data may also include one or more photos of the users and photos of relatives (e.g., ancestors) of the users that are uploaded by the users. A user may authorize the computing server 130 to analyze one or more photos to extract information, such as the user's or relative's appearance traits (e.g., blue eyes, curved hair, etc.), from the photos. The appearance traits and other information extracted from the photos may also be saved in the profile store. In some cases, the computing server may allow users to upload many different photos of the users, their relatives, and even friends. User profile data may also be obtained from other suitable sources, including historical records (e.g., records related to an ancestor), medical records, military records, photographs, other records indicating one or more traits, and other suitable recorded data.

For example, the computing server 130 may present various survey questions to its users from time to time. The responses to the survey questions may be stored at individual profile store 210. The survey questions may be related to various aspects of the users and the users' families. Some survey questions may be related to users' phenotypes, while other questions may be related to environmental factors of the users.

Survey questions may concern health or disease-related phenotypes, such as questions related to the presence or absence of genetic diseases or disorders, inheritable diseases or disorders, or other common diseases or disorders that have a family history as one of the risk factors, questions regarding any diagnosis of increased risk of any diseases or disorders, and questions concerning wellness-related issues such as a family history of obesity, family history of causes of death, etc. The diseases identified by the survey questions may be related to single-gene diseases or disorders that are caused by a single-nucleotide variant, an insertion, or a deletion. The diseases identified by the survey questions may also be multifactorial inheritance disorders that may be caused by a combination of environmental factors and genes. Examples of multifactorial inheritance disorders may include heart disease, Alzheimer's disease, diabetes, cancer, and obesity. The computing server 130 may obtain data on a user's disease-related phenotypes from survey questions about the health history of the user and her family and also from health records uploaded by the user.

Survey questions also may be related to other types of phenotypes such as appearance traits of the users. A survey regarding appearance traits and characteristics may include questions related to eye color, iris pattern, freckles, chin types, finger length, dimple chin, earlobe types, hair color, hair curl, skin pigmentation, susceptibility to skin burn, bitter taste, male baldness, baldness pattern, presence of unibrow, presence of wisdom teeth, height, and weight. A survey regarding other traits also may include questions related to users' taste and smell such as the ability to taste bitterness, asparagus smell, cilantro aversion, etc. A survey regarding traits may further include questions related to users' body conditions such as lactose tolerance, caffeine consumption, malaria resistance, norovirus resistance, muscle performance, alcohol flush, etc. Other survey questions regarding a person's physiological or psychological traits may include vitamin traits and sensory traits such as the ability to sense an asparagus metabolite. Traits may also be collected from historical records, electronic health records and electronic medical records.

The computing server 130 also may present various survey questions related to the environmental factors of users. In this context, an environmental factor may be a factor that is not directly connected to the genetics of the users. Environmental factors may include users' preferences, habits, and lifestyles. For example, a survey regarding users' preferences may include questions related to things and activities that users like or dislike, such as types of music a user enjoys, dancing preference, party-going preference, certain sports that a user plays, video game preferences, etc. Other questions may be related to the users' diet preferences such as like or dislike a certain type of food (e.g., ice cream, egg). A survey related to habits and lifestyle may include questions regarding smoking habits, alcohol consumption and frequency, daily exercise duration, sleeping habits (e.g., morning person versus night person), sleeping cycles and problems, hobbies, and travel preferences. Additional environmental factors may include diet amount (calories, macronutrients), physical fitness abilities (e.g., stretching, flexibility, heart rate recovery), family type (adopted family or not, has siblings or not, lived with extended family during childhood), property and item ownership (has home or rents, has a smartphone or doesn't, has a car or doesn't).

Surveys also may be related to other environmental factors such as geographical, social-economic, or cultural factors. Geographical questions may include questions related to the birth location, family migration history, town, or city of users' current or past residence. Social-economic questions may be related to users' education level, income, occupations, self-identified demographic groups, etc. Questions related to culture may concern users' native language, language spoken at home, customs, dietary practices, etc. Other questions related to users' cultural and behavioral questions are also possible.

For any survey questions asked, the computing server 130 may also ask an individual the same or similar questions regarding the traits and environmental factors of the ancestors, family members, other relatives or friends of the individual. For example, a user may be asked about the native language of the user and the native languages of the user's parents and grandparents. A user may also be asked about the health history of his or her family members.

In addition to storing the survey data in the individual profile store 210, the computing server 130 may store some responses that correspond to data related to genealogical and genetics respectively to genealogy data store 200 and genetic data store 205.

The user profile data, photos of users, survey response data, the genetic data, and the genealogy data may be subject to the privacy and authorization setting of the users to specify any data related to the users that can be accessed, stored, obtained, or otherwise used. For example, when presented with a survey question, a user may select to answer or skip the question. The computing server 130 may present users from time to time information regarding users' selection of the extent of information and data shared. The computing server 130 also may maintain and enforce one or more privacy settings for users in connection with the access of the user profile data, photos, genetic data, and other sensitive data. For example, the user may pre-authorize the access to the data and may change the setting as wished. The privacy settings also may allow a user to specify (e.g., by opting out, by not opting in) whether the computing server 130 may receive, collect, log, or store particular data associated with the user for any purpose. A user may restrict her data at various levels. For example, on one level, the data may not be accessed by the computing server 130 for purposes other than displaying the data in the user's own profile. On another level, the user may authorize anonymization of her data and participate in studies and research conducted by the computing server 130 such as a large-scale genetic study. On yet another level, the user may turn some portions of her genealogy data public to allow the user to be discovered by other users (e.g., potential relatives) and be connected to one or more family trees. Access or sharing of any information or data in the computing server 130 may also be subject to one or more similar privacy policies. A user's data and content objects in the computing server 130 may also be associated with different levels of restriction. The computing server 130 may also provide various notification features to inform and remind users of their privacy and access settings. For example, when privacy settings for a data entry allow a particular user or other entities to access the data, the data may be described as being “visible,” “public,” or other suitable labels, contrary to a “private” label.

In some cases, the computing server 130 may have a heightened privacy protection on certain types of data and data related to certain vulnerable groups. In some cases, the heightened privacy settings may strictly prohibit the use, analysis, and sharing of data related to a certain vulnerable group. In other cases, the heightened privacy settings may specify that data subject to those settings require prior approval for access, publication, or other use. In some cases, the computing server 130 may provide the heightened privacy as a default setting for certain types of data, such as genetic data or any data that the user marks as sensitive. The user may opt in to sharing of those data or change the default privacy settings. In other cases, the heightened privacy settings may apply across the board for all data of certain groups of users. For example, if computing server 130 determines that the user is a minor or has recognized that a picture of a minor is uploaded, the computing server 130 may designate all profile data associated with the minor as sensitive. In those cases, the computing server 130 may have one or more extra steps in seeking and confirming any sharing or use of the sensitive data.

The sample pre-processing engine 215 receives and pre-processes data received from various sources to change the data into a format used by the computing server 130. For genealogy data, the sample pre-processing engine 215 may receive data from an individual via the user interface 115 of the client device 110. To collect the user data (e.g., genealogical and survey data), the computing server 130 may cause an interactive user interface on the client device 110 to display interface elements in which users can provide genealogy data and survey data. Additional data may be obtained from scans of public records. The data may be manually provided or automatically extracted via, for example, optical character recognition (OCR) performed on census records, town or government records, or any other item of printed or online material. Some records may be obtained by digitalizing written records such as older census records, birth certificates, death certificates, etc.

The sample pre-processing engine 215 may also receive raw data from genetic data extraction service server 125. The genetic data extraction service server 125 may perform laboratory analysis of biological samples of users and generate sequencing results in the form of digital data. The sample pre-processing engine 215 may receive the raw genetic datasets from the genetic data extraction service server 125. Most of the mutations that are passed down to descendants are related to single-nucleotide polymorphism (SNP). SNP is a substitution of a single nucleotide that occurs at a specific position in the genome. The sample pre-processing engine 215 may convert the raw base pair sequence into a sequence of genotypes of target SNP sites. Alternatively, the pre-processing of this conversion may be performed by the genetic data extraction service server 125. The sample pre-processing engine 215 identifies autosomal SNPs in an individual's genetic dataset. In some embodiments, the SNPs may be autosomal SNPs. In some embodiments, 700,000 SNPs may be identified in an individual's data and may be stored in genetic data store 205. Alternatively, in some embodiments, a genetic dataset may include at least 10,000 SNP sites. In another embodiment, a genetic dataset may include at least 100,000 SNP sites. In yet another embodiment, a genetic dataset may include at least 300,000 SNP sites. In yet another embodiment, a genetic dataset may include at least 1,000,000 SNP sites. The sample pre-processing engine 215 may also convert the nucleotides into bits. The identified SNPs, in bits or in other suitable formats, may be provided to the phasing engine 220 which phases the individual's diploid genotypes to generate a pair of haplotypes for each user.

The phasing engine 220 phases diploid genetic dataset into a pair of haploid genetic datasets and may perform imputation of SNP values at certain sites whose alleles are missing. An individual's haplotype may refer to a collection of alleles (e.g., a sequence of alleles) that are inherited from a parent.

Phasing may include a process of determining the assignment of alleles (particularly heterozygous alleles) to chromosomes. Owing to sequencing conditions and other constraints, a sequencing result often includes data regarding a pair of alleles at a given SNP locus of a pair of chromosomes but may not be able to distinguish which allele belongs to which specific chromosome. The phasing engine 220 uses a genotype phasing algorithm to assign one allele to a first chromosome and another allele to another chromosome. The genotype phasing algorithm may be developed based on an assumption of linkage disequilibrium (LD), which states that haplotype in the form of a sequence of alleles tends to cluster together. The phasing engine 220 is configured to generate phased sequences that are also commonly observed in many other samples. Put differently, haplotype sequences of different individuals tend to cluster together. A haplotype-cluster model may be generated to determine the probability distribution of a haplotype that includes a sequence of alleles. The haplotype-cluster model may be trained based on labeled data that includes known phased haplotypes from a trio (parents and a child). A trio is used as a training sample because the correct phasing of the child is almost certain by comparing the child's genotypes to the parent's genetic datasets. The haplotype-cluster model may be generated iteratively along with the phasing process with a large number of unphased genotype datasets. The haplotype-cluster model may also be used to impute one or more missing data.

By way of example, the phasing engine 220 may use a directed acyclic graph model such as a hidden Markov model (HMM) to perform the phasing of a target genotype dataset. The directed acyclic graph may include multiple levels, each level having multiple nodes representing different possibilities of haplotype clusters. An emission probability of a node, which may represent the probability of having a particular haplotype cluster given an observation of the genotypes may be determined based on the probability distribution of the haplotype-cluster model. A transition probability from one node to another may be initially assigned to a non-zero value and be adjusted as the directed acyclic graph model and the haplotype-cluster model are trained. Various paths are possible in traversing different levels of the directed acyclic graph model. The phasing engine 220 determines a statistically likely path, such as the most probable path or a probable path that is at least more likely than 95% of other possible paths, based on the transition probabilities and the emission probabilities. A suitable dynamic programming algorithm such as the Viterbi algorithm may be used to determine the path. The determined path may represent the phasing result. U.S. Pat. No. 10,679,729, entitled “Haplotype Phasing Models,” granted on Jun. 9, 2020, describes example embodiments of haplotype phasing. Other example phasing embodiments are described in U.S. Patent Application Publication No. US 2021/0034647, entitled “Clustering of Matched Segments to Determine Linkage of Dataset in a Database,” published on Feb. 4, 2021.

The IBD estimation engine 225 estimates the amount of shared genetic segments between a pair of individuals based on phased genotype data (e.g., haplotype datasets) that are stored in the genetic data store 205. IBD segments may be segments identified in a pair of individuals that are putatively determined to be inherited from a common ancestor. The IBD estimation engine 225 retrieves a pair of haplotype datasets for each individual. The IBD estimation engine 225 may divide each haplotype dataset sequence into a plurality of windows. Each window may include a fixed number of SNP sites (e.g., about 100 SNP sites). The IBD estimation engine 225 identifies one or more seed windows in which the alleles at all SNP sites in at least one of the phased haplotypes between two individuals are identical. The IBD estimation engine 225 may expand the match from the seed windows to nearby windows until the matched windows reach the end of a chromosome or until a homozygous mismatch is found, which indicates the mismatch is not attributable to potential errors in phasing or imputation. The IBD estimation engine 225 determines the total length of matched segments, which may also be referred to as IBD segments. The length may be measured in the genetic distance in the unit of centimorgans (cM). A unit of centimorgan may be a genetic length. For example, two genomic positions that are one cM apart may have a 1% chance during each meiosis of experiencing a recombination event between the two positions. The computing server 130 may save data regarding individual pairs who share a length of IBD segments exceeding a predetermined threshold (e.g., 6 cM), in a suitable data store such as in the genealogy data store 200. U.S. Pat. No. 10,114,922, entitled “Identifying Ancestral Relationships Using a Continuous stream of Input,” granted on Oct. 30, 2018, and U.S. Pat. No. 10,720,229, entitled “Reducing Error in Predicted Genetic Relationships,” granted on Jul. 21, 2020, describe example embodiments of IBD estimation.

Typically, individuals who are closely related share a relatively large number of IBD segments, and the IBD segments tend to have longer lengths (individually or in aggregate across one or more chromosomes). In contrast, individuals who are more distantly related share relatively fewer IBD segments, and these segments tend to be shorter (individually or in aggregate across one or more chromosomes). For example, while close family members often share upwards of 71 cM of IBD (e.g., third cousins), more distantly related individuals may share less than 12 cM of IBD. The extent of relatedness in terms of IBD segments between two individuals may be referred to as IBD affinity. For example, the IBD affinity may be measured in terms of the length of IBD segments shared between two individuals.

Community assignment engine 230 assigns individuals to one or more genetic communities based on the genetic data of the individuals. A genetic community may correspond to an ethnic origin or a group of people descended from a common ancestor. The granularity of genetic community classification may vary depending on embodiments and methods used to assign communities. For example, in some embodiments, the communities may be African, Asian, European, etc. In another embodiment, the European community may be divided into Irish, German, Swedes, etc. In yet another embodiment, the Irish may be further divided into Irish in Ireland, Irish immigrated to America in 1800, Irish immigrated to America in 1900, etc. The community classification may also depend on whether a population is admixed or unadmixed. For an admixed population, the classification may further be divided based on different ethnic origins in a geographical region.

Community assignment engine 230 may assign individuals to one or more genetic communities based on their genetic datasets using machine learning models trained by unsupervised learning or supervised learning. In an unsupervised approach, the community assignment engine 230 may generate data representing a partially connected undirected graph. In this approach, the community assignment engine 230 represents individuals as nodes. Some nodes are connected by edges whose weights are based on IBD affinity between two individuals represented by the nodes. For example, if the total length of two individuals' shared IBD segments does not exceed a predetermined threshold, the nodes are not connected. The edges connecting two nodes are associated with weights that are measured based on the IBD affinities. The undirected graph may be referred to as an IBD network. The community assignment engine 230 uses clustering techniques such as modularity measurement (e.g., the Louvain method) to classify nodes into different clusters in the IBD network. Each cluster may represent a community. The community assignment engine 230 may also determine sub-clusters, which represent sub-communities. The computing server 130 saves the data representing the IBD network and clusters in the IBD network data store 235. U.S. Pat. No. 10,223,498, entitled “Discovering Population Structure from Patterns of Identity-By-Descent,” granted on Mar. 5, 2019, describes example embodiments of community detection and assignment.

The community assignment engine 230 may also assign communities using supervised techniques. For example, genetic datasets of known genetic communities (e.g., individuals with confirmed ethnic origins) may be used as training sets that have labels of the genetic communities. Supervised machine learning classifiers, such as logistic regressions, support vector machines, random forest classifiers, and neural networks may be trained using the training set with labels. A trained classifier may distinguish binary or multiple classes. For example, a binary classifier may be trained for each community of interest to determine whether a target individual's genetic dataset belongs or does not belong to the community of interest. A multi-class classifier such as a neural network may also be trained to determine whether the target individual's genetic dataset most likely belongs to one of several possible genetic communities.

Reference panel sample store 240 stores reference panel samples for different genetic communities. A reference panel sample is a genetic data of an individual whose genetic data is the most representative of a genetic community. The genetic data of individuals with the typical alleles of a genetic community may serve as reference panel samples. For example, some alleles of genes may be over-represented (e.g., being highly common) in a genetic community. Some genetic datasets include alleles that are commonly present among members of the community. Reference panel samples may be used to train various machine learning models in classifying whether a target genetic dataset belongs to a community, determining the ethnic composition of an individual, and determining the accuracy of any genetic data analysis, such as by computing a posterior probability of a classification result from a classifier.

A reference panel sample may be identified in different ways. In some embodiments, an unsupervised approach in community detection may apply the clustering algorithm recursively for each identified cluster until the sub-clusters contain a number of nodes that are smaller than a threshold (e.g., contains fewer than 1000 nodes). For example, the community assignment engine 230 may construct a full IBD network that includes a set of individuals represented by nodes and generate communities using clustering techniques. The community assignment engine 230 may randomly sample a subset of nodes to generate a sampled IBD network. The community assignment engine 230 may recursively apply clustering techniques to generate communities in the sampled IBD network. The sampling and clustering may be repeated for different randomly generated sampled IBD networks for various runs. Nodes that are consistently assigned to the same genetic community when sampled in various runs may be classified as a reference panel sample. The community assignment engine 230 may measure the consistency in terms of a predetermined threshold. For example, if a node is classified to the same community 95% (or another suitable threshold) of the times whenever the node is sampled, the genetic dataset corresponding to the individual represented by the node may be regarded as a reference panel sample. Additionally, or alternatively, the community assignment engine 230 may select N most consistently assigned nodes as a reference panel for the community.

Other ways to generate reference panel samples are also possible. For example, the computing server 130 may collect a set of samples and gradually filter and refine the samples until high-quality reference panel samples are selected. For example, a candidate reference panel sample may be selected from an individual whose recent ancestors are born at a certain birthplace. The computing server 130 may also draw sequence data from the Human Genome Diversity Project (HGDP). Various candidates may be manually screened based on their family trees, relatives' birth location, and other quality control. Principal component analysis may be used to create clusters of genetic data of the candidates. Each cluster may represent an ethnicity. The predictions of the ethnicity of those candidates may be compared to the ethnicity information provided by the candidates to perform further screening.

The ethnicity estimation engine 245 estimates the ethnicity composition of a genetic dataset of a target individual. The genetic datasets used by the ethnicity estimation engine 245 may be genotype datasets or haplotype datasets. For example, the ethnicity estimation engine 245 estimates the ancestral origins (e.g., ethnicity) based on the individual's genotypes or haplotypes at the SNP sites. To take a simple example of three ancestral populations corresponding to African, European and Native American, an admixed user may have nonzero estimated ethnicity proportions for all three ancestral populations, with an estimate such as [0.05, 0.65, 0.30], indicating that the user's genome is 5% attributable to African ancestry, 65% attributable to European ancestry and 30% attributable to Native American ancestry. The ethnicity estimation engine 245 generates the ethnic composition estimate and stores the estimated ethnicities in a data store of computing server 130 with a pointer in association with a particular user.

In some embodiments, the ethnicity estimation engine 245 divides a target genetic dataset into a plurality of windows (e.g., about 1000 windows). Each window includes a small number of SNPs (e.g., 300 SNPs). The ethnicity estimation engine 245 may use a directed acyclic graph model to determine the ethnic composition of the target genetic dataset. The directed acyclic graph may represent a trellis of an inter-window hidden Markov model (HMM). The graph includes a sequence of a plurality of node groups. Each node group, representing a window, includes a plurality of nodes. The nodes represent different possibilities of labels of genetic communities (e.g., ethnicities) for the window. A node may be labeled with one or more ethnic labels. For example, a level includes a first node with a first label representing the likelihood that the window of SNP sites belongs to a first ethnicity and a second node with a second label representing the likelihood that the window of SNPs belongs to a second ethnicity. Each level includes multiple nodes so that there are many possible paths to traverse the directed acyclic graph.

The nodes and edges in the directed acyclic graph may be associated with different emission probabilities and transition probabilities. An emission probability associated with a node represents the likelihood that the window belongs to the ethnicity labeling the node given the observation of SNPs in the window. The ethnicity estimation engine 245 determines the emission probabilities by comparing SNPs in the window corresponding to the target genetic dataset to corresponding SNPs in the windows in various reference panel samples of different genetic communities stored in the reference panel sample store 240. The transition probability between two nodes represents the likelihood of transition from one node to another across two levels. The ethnicity estimation engine 245 determines a statistically likely path, such as the most probable path or a probable path that is at least more likely than 95% of other possible paths, based on the transition probabilities and the emission probabilities. A suitable dynamic programming algorithm such as the Viterbi algorithm or the forward-backward algorithm may be used to determine the path. After the path is determined, the ethnicity estimation engine 245 determines the ethnic composition of the target genetic dataset by determining the label compositions of the nodes that are included in the determined path. U.S. Pat. No. 10,558,930, entitled “Local Genetic Ethnicity Determination System,” granted on Feb. 11, 2020 and U.S. Pat. No. 10,692,587, granted on Jun. 23, 2020, entitled “Global Ancestry Determination System” describe different example embodiments of ethnicity estimation.

The front-end interface 250 displays various results determined by the computing server 130. The results and data may include the IBD affinity between a user and another individual, the community assignment of the user, the ethnicity estimation of the user, phenotype prediction and evaluation, genealogy data search, family tree and pedigree, relative profile and other information. The front-end interface 250 may allow users to manage their profile and data trees (e.g., family trees). The users may view various public family trees stored in the computing server 130 and search for individuals and their genealogy data via the front-end interface 250. The computing server 130 may suggest or allow the user to manually review and select potentially related individuals (e.g., relatives, ancestors, close family members) to add to the user's data tree. The front-end interface 250 may be a graphical user interface (GUI) that displays various information and graphical elements. The front-end interface 250 may take different forms. In one case, the front-end interface 250 may be a software application that can be displayed on an electronic device such as a computer or a smartphone. The software application may be developed by the entity controlling the computing server 130 and be downloaded and installed on the client device 110. In another case, the front-end interface 250 may take the form of a webpage interface of the computing server 130 that allows users to access their family tree and genetic analysis results through web browsers. In yet another case, the front-end interface 250 may provide an application program interface (API).

The tree management engine 260 performs computations and other processes related to users' management of their data trees such as family trees. The tree management engine 260 may allow a user to build a data tree from scratch or to link the user to existing data trees. In some embodiments, the tree management engine 260 may suggest a connection between a target individual and a family tree that exists in the family tree database by identifying potential family trees for the target individual and identifying one or more most probable positions in a potential family tree. A user (target individual) may wish to identify family trees to which he or she may potentially belong. Linking a user to a family tree or building a family may be performed automatically, manually, or using techniques with a combination of both. In an embodiment of an automatic tree matching, the tree management engine 260 may receive a genetic dataset from the target individual as input and search related individuals that are IBD-related to the target individual. The tree management engine 260 may identify common ancestors. Each common ancestor may be common to the target individual and one of the related individuals. The tree management engine 260 may in turn output potential family trees to which the target individual may belong by retrieving family trees that include a common ancestor and an individual who is IBD-related to the target individual. The tree management engine 260 may further identify one or more probable positions in one of the potential family trees based on information associated with matched genetic data between the target individual and DNA test takers in the potential family trees through one or more machine learning models or other heuristic algorithms. For example, the tree management engine 260 may try putting the target individual in various possible locations in the family tree and determine the highest probability position(s) based on the genetic datasets of the target individual and other DNA test takers in the family tree and based on genealogy data available to the tree management engine 260. The tree management engine 260 may provide one or more family trees from which the target individual may select. For a suggested family tree, the tree management engine 260 may also provide information on how the target individual is related to other individuals in the tree. In a manual tree building, a user may browse through public family trees and public individual entries in the genealogy data store 200 and individual profile store 210 to look for potential relatives that can be added to the user's family tree. The tree management engine 260 may automatically search, rank, and suggest individuals for the user conduct manual reviews as the user makes progress in the front-end interface 250 in building the family tree.

As used herein, “pedigree” and “family tree” may be interchangeable and may refer to a family tree chart or pedigree chart that shows, diagrammatically, family information, such as family history information, including parentage, offspring, spouses, siblings, or otherwise for any suitable number of generations and/or people, and/or data pertaining to persons represented in the chart. U.S. Pat. No. 11,429,615, entitled “Linking Individual Datasets to a Database,” granted on Aug. 30, 2022, describes example embodiments of how an individual may be linked to existing family trees.

### Embodiments of Relationship Prediction

Embodiments of relationship prediction systems and methods address shortcomings in the art by predicting a MRCA between a target individual and the match individual, and in embodiments a number of generations between the MRCA and the target individual and match individual, allowing a tailored prediction of a possible relationship between the target individual and the match individual. A target individual may be a user or an individual who is currently being studied. The match individual may be a relative, or in a broader sense, a genetic match. The derived result provides a more intuitive sense of how the target individual and the match individual are related, by narrowing the number of possible relationships and by providing a more-intuitive relation in the form of a MRCA. The relationship prediction embodiments advantageously achieve improved prediction results by utilizing features including age difference between a target individual and the match individual to improve the prediction.

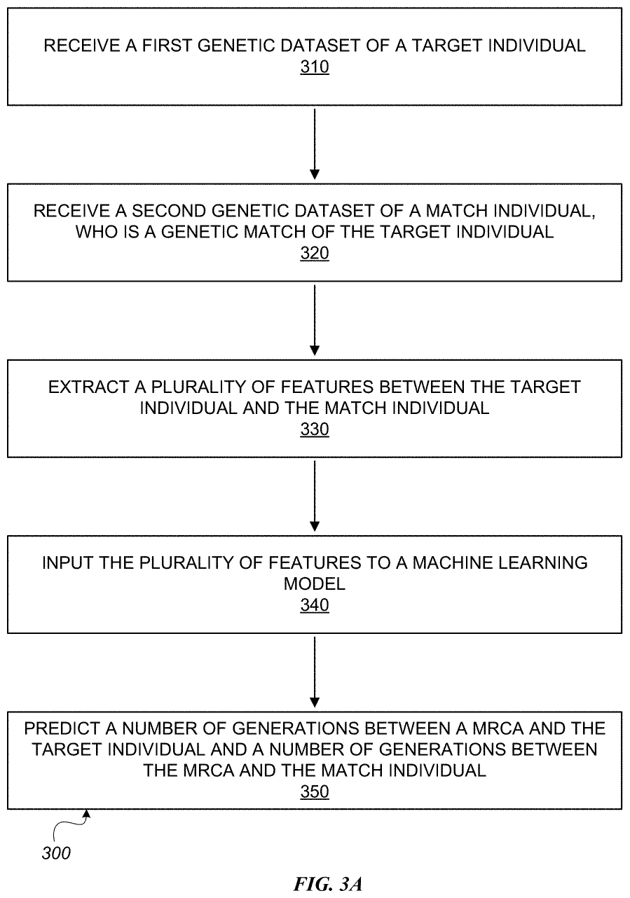

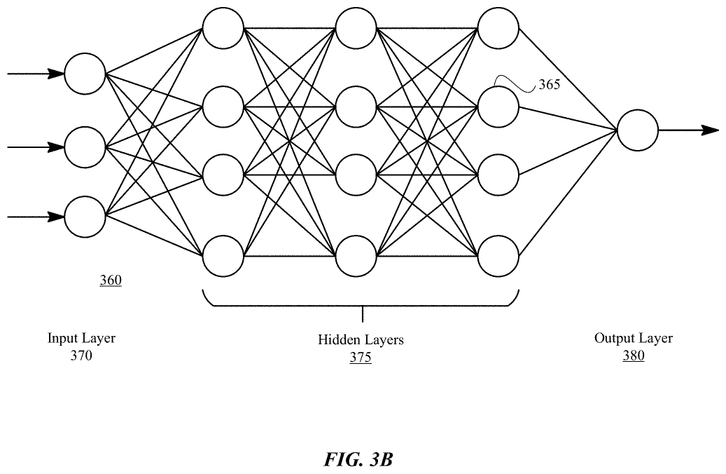

FIG. 3A is a flowchart depicting an example process 300 for determining a number of generations between a MRCA and a target individual and the MRCA and a match individual. The process 300 may be performed by computing devices such as the computing server 130. For example, the tree management engine 260 may use the process 300 to suggest one or more proposed family trees of possible placements, within family trees, of the user and the target individual, to a user. The process 300 may be embodied as a software algorithm that may be stored as computer instructions that are executable by one or more processors. The instructions, when executed by the processors, cause the processors to perform various steps in the process 300. In various embodiments, the process 300 may include additional, fewer, or different steps in any suitable orders. While various steps in process 300 may be discussed with the use of computing server 130, each step may be performed by a different computing device.

The process 300 includes a step 310 of receiving a first genetic dataset of a target individual. In some embodiments, a target individual is a person that has genetic data stored in the computing server 130. The target individual may or may not (e.g., a new user) have a family tree stored on the computing server 130. In some embodiments, the target individual may submit a DNA sample that is processed to be genetic data or the computing server may otherwise acquire the genetic data of the target individual. Receiving the first genetic dataset may require using the genetic data extraction service server 125 to extract genetic data for the target individual. In some embodiments, the genealogy data store 200 or genetic data store 205 contain the first genetic dataset of the target individual.

The process 300 includes a step 320 of receiving a second genetic dataset of a match individual, who may be a genetic match of the target individual or a match that is defined by other criteria. The match individual may be identified based on identity by descent (IBD) matched segments with the target individual, using the IBD estimation engine 225. Identifying potential genetic matches includes identifying a possible relationship between the matches based on factors including, but not limited to, number of cM shared, number of segments shared, a number of IBD segments in the first dataset and second dataset, etc. Those matches may be referred to as IBD matches. Details for identifying a match individual using the IBD estimation engine are further described regarding FIG. 2.

For the purpose of the process 300, a match individual at this stage may be referred to as a candidate match individual because the predicted relationship between the target individual and the candidate match individual is further evaluated in the process 300. While genetic match is used as the primary example in the process 300, other ways, such as using manual suggestion by other users, automatic suggestion based on the tree management engine 260, or genealogy data such as historical records, may also be used to define a match.

Among the potential matches such as genetic matches, a close match (such as the closest match based on a highest number of cM or IBD segments shared) may be further analyzed. An example of a close match may be a third cousin. The computing server 130 may identify a close match who is associated with a family tree stored in the computing server 130. The computing server 130 may retrieve the associated family tree. The family tree contains one or more nodes (representing persons) connected by edges (representing relationships between the persons). In some embodiments, one or more nodes of the retrieved family tree that have associated genetic data are identified. Such steps are described in at least U.S. Pat. No. 11,429,615, granted Aug. 30, 2022, and incorporated herein in its entirety by reference.

With continued reference to FIG. 3A, the process 300 can include an additional step 330 of extracting a plurality of features between the target individual and the match individual. The plurality of features may include a MRCA, number of cM shared, birth years, a number of segments shared, and/or age difference between the target individual and the match individual, in some embodiments. Alternatively, or additionally, the plurality of features between the target individual and the match individual may include one or more genetic features shared between the first and second genetic datasets and an age difference between the target individual and the match individual. The age difference may be ascertained using user input, associated family tree profiles, historical records, or otherwise.

The process 300 can include a step 340 of inputting the plurality of features to a machine learning model. The machine learning model may be trained on training samples. In embodiments, each training sample may include an age difference between a pair of matched individuals, cM shared between the pair, and a number of shared segments between the pair. The shared segments between the pair may include IBD shared segments in the first and second datasets, or a number of shared DNA segments. Training the machine learning model may include receiving training samples that comprise age differences between pairs of matched individuals and known generation data, e.g. a number of generations between the matched individuals and a MRCA and/or each other. The training samples may be input to the machine learning model to generate predicted generation numbers. Predicted generation numbers are compared to known generation data in the training samples, in accordance with some embodiments. The weights of the machine learning model are adjusted based on the comparison between predicted generation numbers and known generation data.

In some embodiments, the process 300 further includes a step 350 of predicting a number of generations between a MRCA and the target individual and a number of generations between the MRCA and the match individual. In some embodiments, the number of generations between the MRCA and each individual is the same number. The predicted number of generations between the MRCA and the target individual and the number of generations between the MRCA and the match individual may be used to generate and/or filter predicted relationships between the target individual and the match individual. The number of generations may be used in combination with the cM shared between the target individual and the match individual, the number of shared DNA segments, and the age difference between the target individual and the match individual to predict relationships for two individuals. In some embodiments, alternative combinations of genetic features are used in combination with the predicted number of generations between the MRCA and the target individual and the number of generations between the MRCA and the match individual to predict a relationship between the target and match individuals.

### Example Machine Learning Models

In various embodiments, a wide variety of machine learning techniques may be used. Examples include different forms of supervised learning, unsupervised learning, and semi-supervised learning such as decision trees, support vector machines (SVMs), regression, Bayesian networks, and genetic algorithms. Deep learning techniques such as neural networks, including convolutional neural networks (CNN), recurrent neural networks (RNN) and long short-term memory networks (LSTM), may also be used. For example, various relationship predictions described in process 300, genetic matching, and other processes may apply one or more machine learning and deep learning techniques.

In various embodiments, the training techniques for a machine learning model may be supervised, semi-supervised, or unsupervised. In supervised learning, the machine learning models may be trained with a set of training samples that are labeled. For example, for a machine learning model trained to predict relationships between a target individual and a match individual in a database, the training samples may be pairs of individuals with known genetic relationships. The labels for each training sample may be binary or multi-class. In training a machine learning model for predicting relationships between individuals, the training labels may include a positive label that indicates a likely familial relationship and a negative label that indicates an unlikely or impossible familial relationship. In some embodiments, the training labels may also be multi-class such as level of relation between individuals (M1, M2, M3, etc.).

By way of example, the training set may include the proposed family trees for multiple previous target individuals with a known correct family tree. Each training sample in the training set may correspond to a past and the corresponding outcome may serve as the label for the sample. A training sample may be represented as a feature vector that includes multiple dimensions. Each dimension may include data of a feature, which may be a quantized value of an attribute that describes the past record. For example, in a machine learning model that is used to predict relationship between individuals, the features in a feature vector may include number of cM shared, generations from an MRCA, age difference, and/or various features described throughout this disclosure. In various embodiments, certain pre-processing techniques may be used to normalize the values in different dimensions of the feature vector.

In some embodiments, an unsupervised learning technique may be used. The training samples used for an unsupervised model may also be represented by feature vectors, but may not be labeled. Various unsupervised learning techniques such as clustering may be used in determining similarities among the feature vectors, thereby categorizing the training samples into different clusters. In some cases, the training may be semi-supervised with a training set having a mix of labeled samples and unlabeled samples.

A machine learning model may be associated with an objective function, which generates a metric value that describes the objective goal of the training process. The training process may intend to reduce the error rate of the model in generating predictions. In such a case, the objective function may monitor the error rate of the machine learning model. In a model that generates predictions, the objective function of the machine learning algorithm may be the training error rate when the predictions are compared to the actual labels. Such an objective function may be called a loss function. Other forms of objective functions may also be used, particularly for unsupervised learning models whose error rates are not easily determined due to the lack of labels. In some embodiments, in relationship prediction, the objective function may correspond to determining potential relationships between a target individual and a match individual. In various embodiments, the error rate may be measured as cross-entropy loss, L1 loss (e.g., the sum of absolute differences between the predicted values and the actual value), L2 loss (e.g., the sum of squared distances), or otherwise.

Referring to FIG. 3B, a structure of an example neural network 360 is illustrated, in accordance with some embodiments. The neural network 360 may receive an input and generate an output. The input may be the feature vector of a training sample in the training process and the feature vector of an actual case when the neural network is making an inference. The output may be the prediction, classification, or another determination performed by the neural network. The neural network 360 may include different kinds of layers, such as convolutional layers, pooling layers, recurrent layers, fully connected layers, and custom layers. A convolutional layer convolves the input of the layer (e.g., an image) with one or more kernels to generate different types of images that are filtered by the kernels to generate feature maps. Each convolution result may be associated with an activation function. A convolutional layer may be followed by a pooling layer that selects the maximum value (max pooling) or average value (average pooling) from the portion of the input covered by the kernel size. The pooling layer reduces the spatial size of the extracted features. In some embodiments, a pair of convolutional layer and pooling layer may be followed by a recurrent layer that includes one or more feedback loops. The feedback may be used to account for spatial relationships of the features in an image or temporal relationships of the objects in the image. The layers may be followed by multiple fully connected layers that have nodes connected to each other. The fully connected layers may be used for classification and object detection. In one embodiment, one or more custom layers may also be presented for the generation of a specific format of output. For example, a custom layer may be used for image segmentation for labeling pixels of an image input with different segment labels.

The order of layers and the number of layers of the neural network 360 may vary in different embodiments. In various embodiments, a neural network 360 includes one or more layers 370, 375, and 380, including an input layer 370, hidden layers 375, and an output layer 380, but may or may not include any pooling layer or recurrent layer. If a pooling layer is present, not all convolutional layers are always followed by a pooling layer. A recurrent layer may also be positioned differently at other locations of the CNN. For each convolutional layer, the sizes of kernels (e.g., 3×3, 5×5, 7×7, etc.) and the numbers of kernels allowed to be learned may be different from other convolutional layers.

A machine learning model may include certain layers, nodes 365, kernels and/or coefficients. Training of a neural network, such as the neural network 360, may include forward propagation and backpropagation. Each layer in a neural network may include one or more nodes, which may be fully or partially connected to other nodes in adjacent layers. In forward propagation, the neural network performs the computation in the forward direction based on outputs of a preceding layer. The operation of a node may be defined by one or more functions. The functions that define the operation of a node may include various computation operations such as convolution of data with one or more kernels, pooling, recurrent loop in RNN, various gates in LSTM, etc. The functions may also include an activation function that adjusts the weight of the output of the node. Nodes in different layers may be associated with different functions.

Training of a machine learning model may include an iterative process that includes iterations of making determinations, monitoring performance of the machine learning model using the objective function, and backpropagation to adjust the weights (e.g., weights, kernel values, coefficients) in various nodes 365. For example, a computing device may receive a training set that includes two individuals and a corresponding number of cM shared, shared genetic segments, age difference, ethnicity, and a MRCA. Each training sample in the training set may be assigned with labels indicating a level of relationships between pairs of individuals. The computing device, in a forward propagation, may use the machine learning model to generate predicted relationships between pairs of individuals. The computing device may compare the predicted relationships between pairs of individuals with the labels of the training sample. The computing device may adjust, in a backpropagation, weights of the machine learning model based on the comparison.

By way of example, each of the functions in the neural network may be associated with different coefficients (e.g., weights and kernel coefficients) that are adjustable during training. In addition, some of the nodes in a neural network may also be associated with an activation function that decides the weight of the output of the node in forward propagation. Common activation functions may include step functions, linear functions, sigmoid functions, hyperbolic tangent functions (tanh), and rectified linear unit functions (ReLU). After an input is provided into the neural network and passes through a neural network in the forward direction, the results may be compared to the training labels or other values in the training set to determine the neural network's performance. The process of prediction may be repeated for other images in the training sets to compute the value of the objective function in a particular training round. In turn, the neural network performs backpropagation by using gradient descent such as stochastic gradient descent (SGD) to adjust the coefficients in various functions to improve the value of the objective function.

Multiple rounds of forward propagation and backpropagation may be performed. Training may be completed when the objective function has become sufficiently stable (e.g., the machine learning model has converged) or after a predetermined number of rounds for a particular set of training samples. The trained machine learning model can be used for performing relationship prediction or another suitable task for which the model is trained.

Illustrations with Family Trees

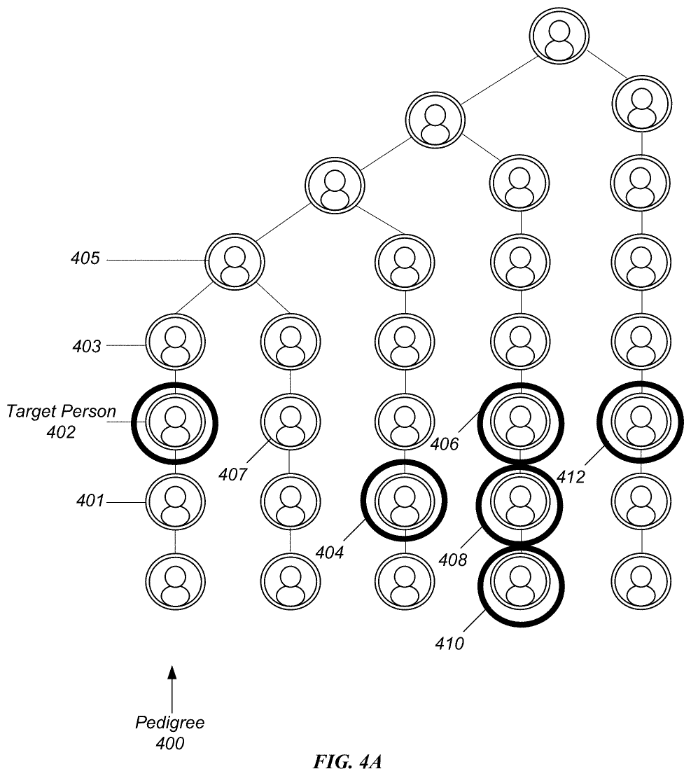

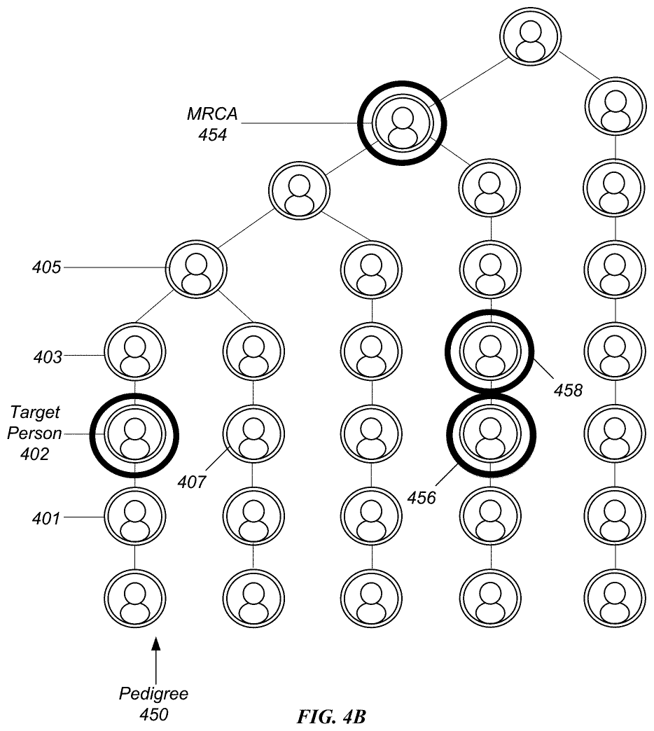

Turning to FIG. 4A, a family tree 400 is shown with a target individual (e.g., a user) 402 located therewithin. Also represented are the target individual's parent 403, child 401, grandparent 405, first cousin 407, and so on. The target individual 402 may submit a DNA sample to a genealogical research service, and a relative may be identified based on IBD, for example. However, if the relationship is attenuated, e.g., M3+, there may be multiple plausible candidates within the family tree 400 for how two people are related. For example, the match individual may be related to the target individual 402 as a second cousin once removed 404, as a third cousin 406, as a third cousin once removed 408, as a third cousin twice removed 410, or as a fourth cousin 412. Not only are such relationships bereft of emotional meaning or intuitive sense to a user, this broad range of possible relationships makes the predicted relationship highly uncertain. Users are unlikely to engage emotionally with such a prediction or to trust its accuracy.

Turning now to FIG. 4B, a family tree 450 is shown with the target individual 402 and a MRCA 454 common to the target individual 402 and a most likely candidate relationship 456 with a target individual. For example, the MRCA 454 is the most recently shared ancestor between the user 402 and the most likely candidate relationship 456 by which the user 402 and their relative 456 are connected. A second-most likely candidate 458 may also be highlighted. This view advantageously allows a user to easily see a most likely relationship between themselves and their relative, as well as understanding a more-intuitive connection, e.g., that they are related through their great-grandparent, for example. This narrows the range of possible relationships, provides for more-accurate predictions generally, and enhances the user experience. The connection, further, is more intuitive for a user, as they are able to understand that they are related to this particular relative through the MRCA, e.g. a great-great grandmother, which they are more likely to be aware of, understand, and feel connected to.

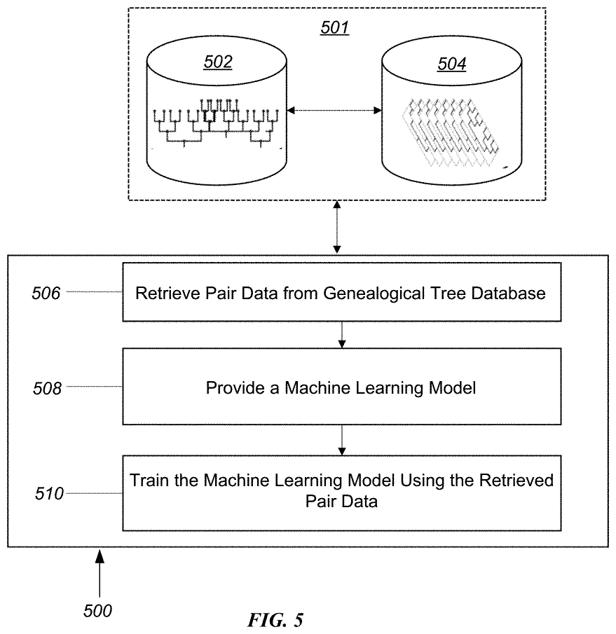

Turning now to FIG. 5, a method 500 of training a machine learning model for relationship predictions according to embodiments is shown. The method 500 includes a step 506 of receiving data from a genealogical tree database. The genealogical tree database may be a genealogical tree database 501 comprising a tree database 504 and a cluster database 502, in which overlapping individuals in distinct trees are stitched together into clusters using entity resolution or other techniques. The use of a genealogical tree database advantageously allows for accessing a larger volume and a higher quality of data than would otherwise be possible.

For example, the genealogical tree database 501, by comprising a cluster database 502 comprising nodes representing individuals and edges representing connections between individuals, may allow for access to data on relationships between people on a scale that would not be possible relying on information supplied by users alone, as users are inherently limited in their understanding of their own family history details. The stitching together of individuals represented in different trees allows for leveraging the details about such individuals provided separately by different users who are separately privy to such details but who may not have had opportunity to share such details with each other if, in fact, they are even aware of one another's existence.

In some embodiments, over 30,000 pairs of individuals, in particular embodiments over 35,000 pairs, are retrieved from the genealogical tree database 501. While 30,000+ pairs are described, it will be appreciated that the disclosure is not limited thereto, and that any suitable source, type, and quantity of data may be used where supervised learning methods are utilized.

Each individual represented by each pair may be selected based on having both DNA samples and family trees associated therewith. This allows for generating labels for the data based on a verifiable MRCA that the individuals in the pair share as determined through the pertinent family trees, as well as genetic match information, such as cM shared, number of shared segments, etc. Additional data, such as birth dates and/or estimated birth dates, may likewise be obtained. In some embodiments, the pair data or components thereof are retrieved from a stitched genealogical tree database in which the family trees of each individual in each pair are resolved such that the relationship between the individuals in the pair and their shared MRCA are known or confidently predicted.

A step 508 includes providing a machine learning model. The machine learning model may be one model, several models, a concatenation of models, or otherwise. The machine learning model may be a model such as a classifier model, such as a k-nearest neighbors classifier, a logistic regression-based classifier, a decision tree classifier, an extra tree classifier, an extremely randomized trees classifier, a radius neighbors classifier, a random forest classifier, modifications and/or combinations thereof, or any other suitable approach.

A step 510 includes training the machine learning model using the retrieved pair data. The machine learning model may be trained in any suitable manner using the labels extracted from the training data such that, upon receiving features including, e.g., an age difference between a target and a match, cM between the target and the match, and/or a number of shared segments between the target and the match, the model is able to predict a number of generations between the MRCA and each of the target and the match.

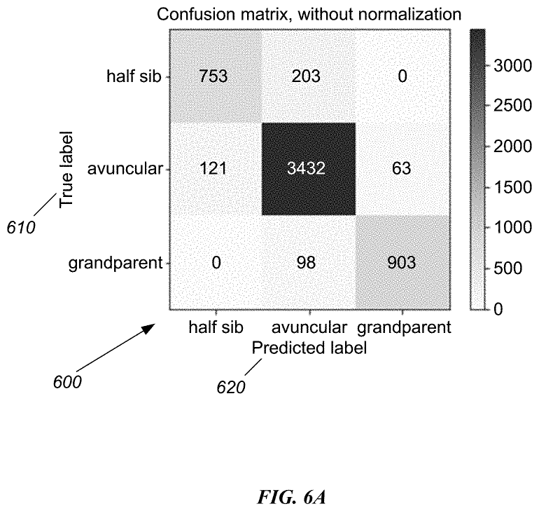

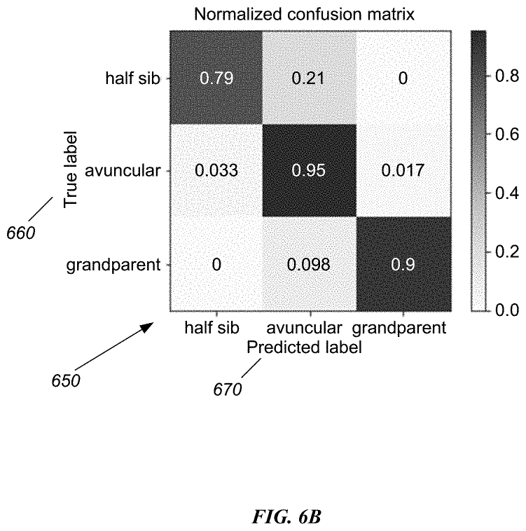

Turning now to FIGS. 6A and 6B, a confusion matrix, without and with normalization, respectively, is depicted for predictions in the M3 relationship category. As seen in FIGS. 6A and 6B, the confusion matrix 600, 650 between the true labels 610, 660 and the predicted labels 620, 670 indicate highly successful predictions for relationships that are notoriously difficult to distinguish on the basis of cM and shared segments alone, e.g., grandparent/avuncular/half-sibling relationships, as on the basis of shared DNA alone grandparent, avuncular, and half-sibling relationships have substantial overlap in terms of DNA shared. That is, the relationship prediction embodiments of the disclosure advantageously facilitate accurately distinguishing between half-sibling relationships, grandparent relationships, and avuncular relationships, as half-sibling relationships are, using existing methods, indistinguishable from grandparent and avuncular relationships on the basis of shared cM and the number of shared segments.

Grandparent relationships are correctly predicted 90% of the time, and avuncular relationships are correctly predicted 95% of the time using the relationship prediction embodiments. It is thought that the accuracy of half-sibling prediction (79%) lags the accuracy of grandparent and avuncular predictions due to the wide range of ages between half siblings in reality, which can be as wide-ranging as avuncular age differences. Nevertheless, 79% accuracy is a substantial improvement over previous attempts to distinguish half-sibling relationships from grandparent and/or avuncular relationships.

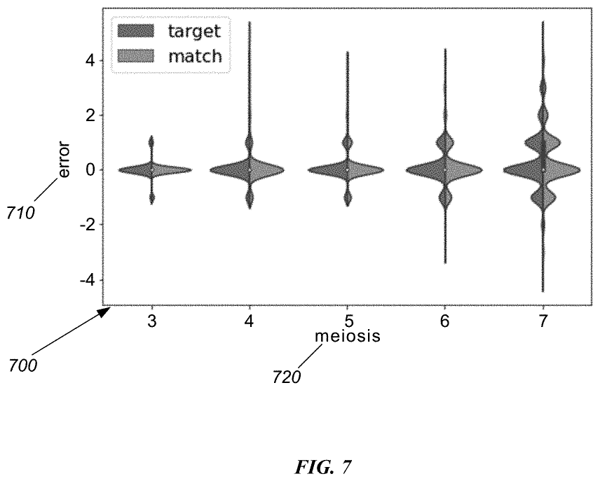

Turning now to FIG. 7, errors 710 for targets and matches at meiosis levels 720 M3-M7 using the disclosed embodiments are shown and described. As seen in graph 700, predictions are mostly within ±1 of truth. That is, shown in FIG. 7 are the predicted generations between the target and the MRCA minus the true number of generations between the target and the MRCA on the left, and the predicted generations between the match and the MRCA minus the true number of generations between the match and the MRCA on the right. As seen, the vast majority of predictions have no error, particularly in M3-M5 but also in M6 and M7 predictions. Even errors that begin to increase at more-attenuated (and harder-to-predict) relationships like M7 still remain clustered largely within ±1 of truth.

Further, the top two predictions are mostly within ±1 of truth, as shown in Tables 2 and 3 below, which correspond respectively to the target and the match. This points out the ability of the disclosed relationship prediction embodiments to accurately and reliably predict a correct relationship using the top two candidate or predicted relationships, even at highly attenuated relationships like M6 and M7. This is a substantial improvement over existing approaches which provide many possible relationships with no ability for a user to effectively rank the proposed relationships. The error is shown in terms of the predicted generations between the target or match and the MRCA minus the true number of generations between the target or match and the MRCA, respectively.

TABLE 2

| Error | 0 | ±1 | ≥2 | Total Samples |
|---|---|---|---|---|
| Meiosis Level | | | | |
| 4 | 96.5% | 3.3% | 0.2% | 3009 |
| 5 | 95.5% | 4.0% | 0.5% | 6298 |
| 6 | 90.5% | 7.9% | 1.6% | 11934 |
| 7 | 72.1% | 19.8% | 8.1% | 63067 |

TABLE 3

| Error | 0 | ±1 | ≥2 | Total Samples |
|---|---|---|---|---|
| Meiosis Level | | | | |
| 4 | 96.5% | 3.2% | 0.3% | 3009 |
| 5 | 95.0% | 4.5% | 0.5% | 6298 |
| 6 | 90.5% | 7.7% | 4.4% | 11934 |
| 7 | 71.8% | 20.6% | 7.7% | 63067 |

Further, the single top prediction is mostly within ±1 of truth, as shown in Tables 4 and 5 below, which correspond respectively to the target and the match. This points up the ability of the disclosed relationship-prediction embodiments to accurately and reliably predict a correct relationship using only the top candidate or predicted relationship. In some embodiments, the Meiosis Level 3 prediction was determined using logistic regression and the Meiosis Level 4-7 predictions were determined using k-nearest neighbors.

TABLE 4

| Error | 0 | ±1 | ≥2 | Total Samples |
|---|---|---|---|---|
| Meiosis Level | | | | |
| 3 | 88.9% | 11.1% | 0.0% | 172 |
| 4 | 83.9% | 15.1% | 1% | 199 |
| 5 | 82.5% | 15.9% | 1.6% | 458 |
| 6 | 70.0% | 26.1% | 4.0% | 753 |
| 7 | 50.0% | 37.6% | 12.4% | 3423 |

TABLE 5

| Error | 0 | ±1 | ≥2 | Total Samples |
|---|---|---|---|---|
| Meiosis Level | | | | |
| 3 | 91.9% | 8.1% | 0.0% | 172 |
| 4 | 83.4% | 14.6% | 2.0% | 199 |
| 5 | 80.1% | 18.4% | 1.5% | 458 |
| 6 | 68.9% | 26.7% | 4.4% | 753 |
| 7 | 48.4% | 38.6% | 13.0% | 3423 |

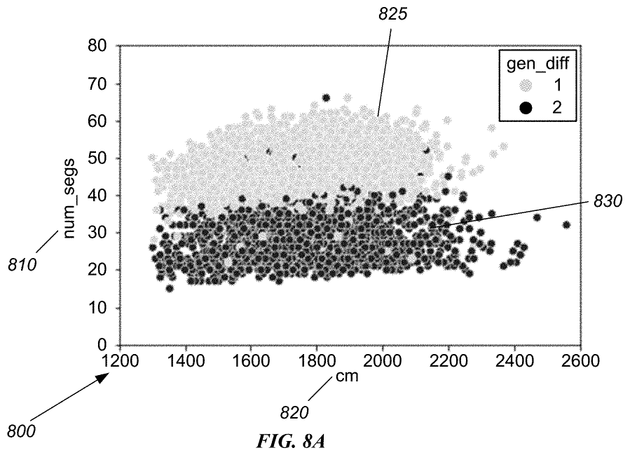

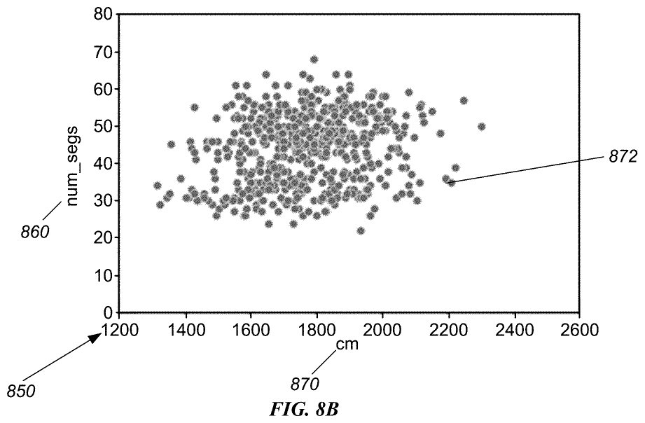

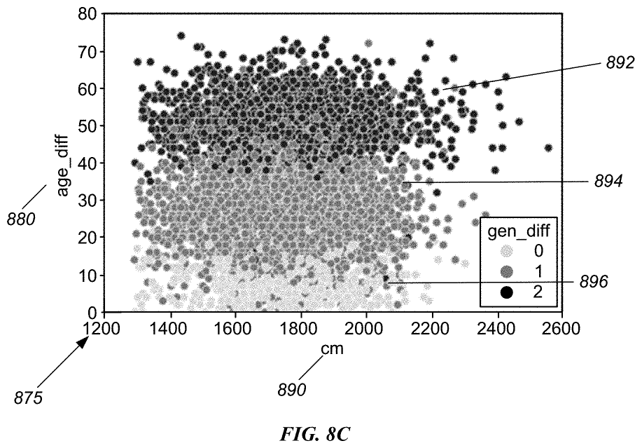

It has been surprisingly found that, in some embodiments, the use of features such as number of shared segments and cM may readily distinguish, at the M3 level, avuncular and grandparent relationships, as seen in FIG. 8A. A graph 800 shows samples plotted as a function of a number of shared segments 810 and cM shared 820. As seen, predictions regarding a number of generations between the MRCA and the target individual or match individual are readily distinguishable, with one-generation predictions 825 (pertaining to avuncular relationships) above and two-generation predictions 830 (pertaining to grandparent relationships) below. That is, the one-generation predictions 825 correspond to avuncular relationships whereas the two-generation predictions 830 correspond to grandparent relationships. cM shared and number of shared segments therefore allow for distinguishing such one- and two-generation relationships at the M3 level.

Turning now to FIG. 8B, however, the wide age distribution of half-sibling relationships makes these relationships more difficult to separate, and therefore more difficult to predict. As seen in a graph 850 in which half-sibling samples are plotted against number of shared segments 860 and cM shared 870, the half-sibling segments 872 are distributed such that they overlap the distribution of the avuncular and grandparent samples 825, 830 above.

It has been surprisingly found, however, that by generating predictions using a combination of cM shared, number of shared segments, and age difference between the target and match as features, half-sibling relationships can be more readily distinguished from grandparent and avuncular relationships, as shown in FIG. 8C. The graph 875, plotted against age difference 880 and cM shared 890, shows that two-generation relationships 892, one-generation relationships 894, and zero-generation relationships 896 can be distinguished within the M3 category of relationships as closely clustered strata. As above, the two-generation relationships 892 correspond to grandparent relationships, one-generation relationships 894 correspond to avuncular relationships, and zero-generation relationships 896 correspond to half-sibling relationships.

That is, it has been surprisingly found that there is a synergistic effect of providing the unique combination of cM shared, number of shared segments, and age difference between a target and a particular match as features for a classification procedure, such as a multilabel-multiclass classification procedure. This allows for distinguishing between otherwise difficult-to-distinguish, if not indistinguishable, potential relationships.

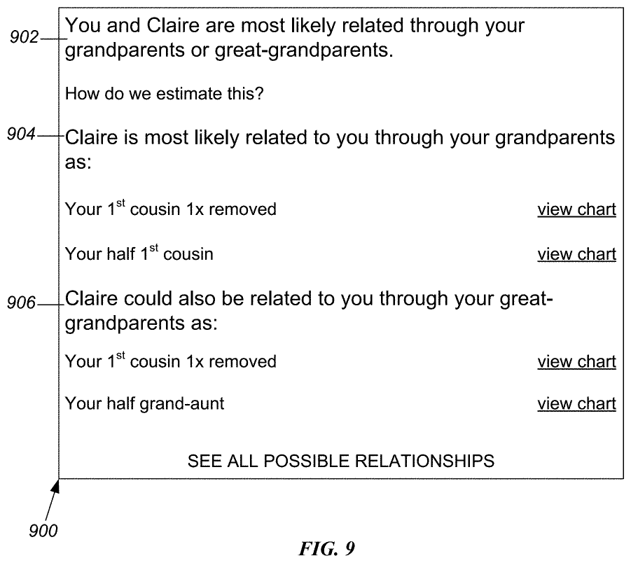

FIG. 9 illustrates a user interface 900 for displaying predicted relationship results. In contrast to an approach where a family tree is shown with many possible relationships, undistinguished by likelihood, the user interface 900 shows a likely MRCA 902 as well as most-likely and second-most likely relationships 904, 906 based on the MRCA. This has been found to provide a more-intuitive and more-focused prediction for a user. The user interface 900 is enabled by the relationship prediction approach described herein.

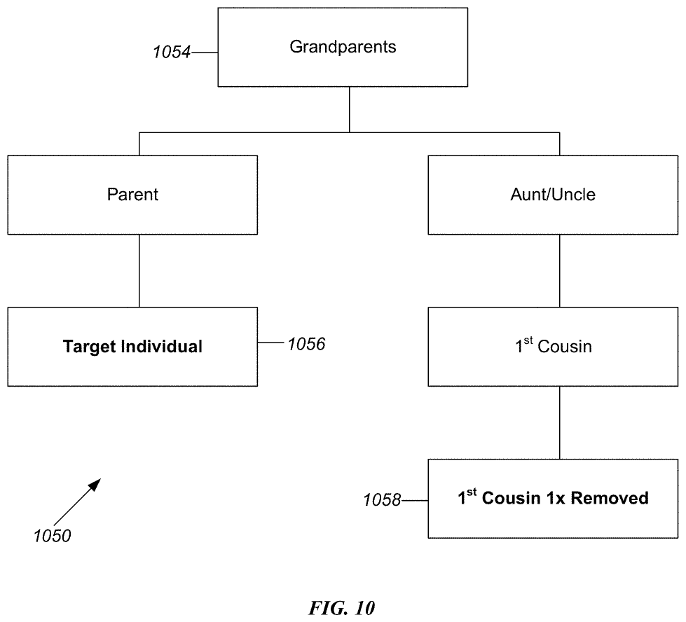

Turning to FIG. 10, a user interface 1050 is shown, where a family tree shows a MRCA 1054, the target individual 1056, and a match individual 1058. The family tree may be generated in an embodiment based on a most-likely prediction of MRCA and a relationship between the target and match individual. This provides an intuitive, simple, and rewarding experience for a user of a genealogical and/or genetic research service. Furthermore, the user interface 1050 advantageously educates a user about the nature of their relationships with a particular match in simple and memorable terms.

### Computing Machine Architecture

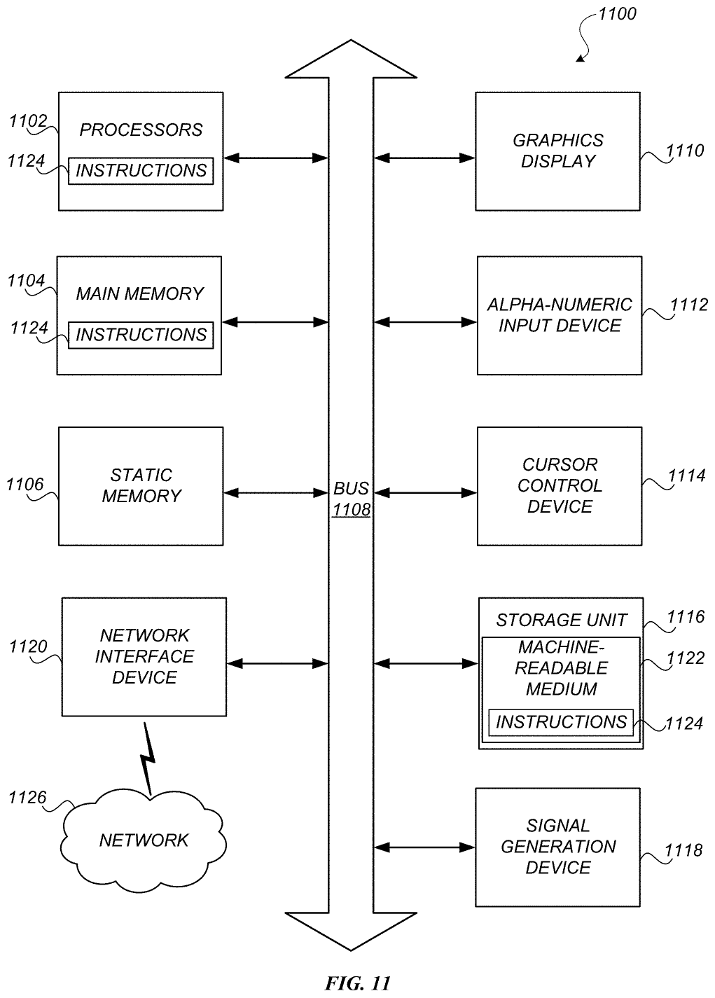

FIG. 11 is a block diagram illustrating components of an example computing machine that is capable of reading instructions from a computer-readable medium and execute them in a processor (or controller). A computer described herein may include a single computing machine shown in FIG. 11, a virtual machine, a distributed computing system that includes multiple nodes of computing machines shown in FIG. 11, or any other suitable arrangement of computing devices.

By way of example, FIG. 11 shows a diagrammatic representation of a computing machine in the example form of a computer system 1100 within which instructions 1124 (e.g., software, source code, program code, expanded code, object code, assembly code, or machine code), which may be stored in a computer-readable medium for causing the machine to perform any one or more of the processes discussed herein may be executed. In some embodiments, the computing machine operates as a standalone device or may be connected (e.g., networked) to other machines. In a networked deployment, the machine may operate in the capacity of a server machine or a client machine in a server-client network environment, or as a peer machine in a peer-to-peer (or distributed) network environment.

The structure of a computing machine described in FIG. 11 may correspond to any software, hardware, or combined components shown in FIGS. 1 and 2, including but not limited to, the client device 110, the computing server 130, and various engines, interfaces, terminals, and machines shown in FIG. 2. While FIG. 11 shows various hardware and software elements, each of the components described in FIGS. 1 and 2 may include additional or fewer elements.

By way of example, a computing machine may be a personal computer (PC), a tablet PC, a set-top box (STB), a personal digital assistant (PDA), a cellular telephone, a smartphone, a web appliance, a network router, an internet of things (IoT) device, a switch or bridge, or any machine capable of executing instructions 1124 that specify actions to be taken by that machine. Further, while only a single machine is illustrated, the term “machine” and “computer” may also be taken to include any collection of machines that individually or jointly execute instructions 1124 to perform any one or more of the methodologies discussed herein.

The example computer system 1100 includes one or more processors 1102 such as a CPU (central processing unit), a GPU (graphics processing unit), a TPU (tensor processing unit), a DSP (digital signal processor), a system on a chip (SOC), a controller, a state equipment, an application-specific integrated circuit (ASIC), a field-programmable gate array (FPGA), or any combination of these. Parts of the computing system 1100 may also include a memory 1104 that store computer code including instructions 1124 that may cause the processors 1102 to perform certain actions when the instructions are executed, directly or indirectly by the processors 1102. Instructions can be any directions, commands, or orders that may be stored in different forms, such as equipment-readable instructions, programming instructions including source code, and other communication signals and orders. Instructions may be used in a general sense and are not limited to machine-readable codes. One or more steps in various processes described may be performed by passing through instructions to one or more multiply-accumulate (MAC) units of the processors.

One and more methods described herein improve the operation speed of the processors 1102 and reduces the space required for the memory 1104. For example, the database processing techniques and machine learning methods described herein reduce the complexity of the computation of the processors 1102 by applying one or more novel techniques that simplify the steps in training, reaching convergence, and generating results of the processors 1102. The algorithms described herein also reduces the size of the models and datasets to reduce the storage space requirement for memory 1104.

The performance of certain operations may be distributed among more than one processor, not only residing within a single machine, but deployed across a number of machines. In some example embodiments, the one or more processors or processor-implemented modules may be located in a single geographic location (e.g., within a home environment, an office environment, or a server farm). In other example embodiments, one or more processors or processor-implemented modules may be distributed across a number of geographic locations. Even though in the specification or the claims may refer some processes to be performed by a processor, this should be construed to include a joint operation of multiple distributed processors.

The computer system 1100 may include a main memory 1104, and a static memory 1106, which are configured to communicate with each other via a bus 1108. The computer system 1100 may further include a graphics display unit 1110 (e.g., a plasma display panel (PDP), a liquid crystal display (LCD), a projector, or a cathode ray tube (CRT)). The graphics display unit 1110, controlled by the processors 1102, displays a graphical user interface (GUI) to display one or more results and data generated by the processes described herein. The computer system 1100 may also include alphanumeric input device 1112 (e.g., a keyboard), a cursor control device 1114 (e.g., a mouse, a trackball, a joystick, a motion sensor, or other pointing instruments), a storage unit 1116 (a hard drive, a solid-state drive, a hybrid drive, a memory disk, etc.), a signal generation device 1118 (e.g., a speaker), and a network interface device 1120, which also are configured to communicate via the bus 1108.

The storage unit 1116 includes a computer-readable medium 1122 on which is stored instructions 1124 embodying any one or more of the methodologies or functions described herein. The instructions 1124 may also reside, completely or at least partially, within the main memory 1104 or within the processor 1102 (e.g., within a processor's cache memory) during execution thereof by the computer system 1100, the main memory 1104 and the processor 1102 also constituting computer-readable media. The instructions 1124 may be transmitted or received over a network 1126 via the network interface device 1120.

While computer-readable medium 1122 is shown in an example embodiment to be a single medium, the term “computer-readable medium” should be taken to include a single medium or multiple media (e.g., a centralized or distributed database, or associated caches and servers) able to store instructions (e.g., instructions 1124). The computer-readable medium may include any medium that is capable of storing instructions (e.g., instructions 1124) for execution by the processors (e.g., processors 1102) and that cause the processors to perform any one or more of the methodologies disclosed herein. The computer-readable medium may include, but not be limited to, data repositories in the form of solid-state memories, optical media, and magnetic media. The computer-readable medium does not include a transitory medium such as a propagating signal or a carrier wave.

### Additional Considerations

The foregoing description of the embodiments has been presented for the purpose of illustration; it is not intended to be exhaustive or to limit the patent rights to the precise forms disclosed. Persons skilled in the relevant art can appreciate that many modifications and variations are possible in light of the above disclosure.

Any feature mentioned in one claim category, e.g., method, can be claimed in another claim category, e.g., computer program product, system, storage medium, as well. The dependencies or references back in the attached claims are chosen for formal reasons only. However, any subject matter resulting from a deliberate reference back to any previous claims (in particular multiple dependencies) can be claimed as well, so that any combination of claims and the features thereof is disclosed and can be claimed regardless of the dependencies chosen in the attached claims. The subject matter may include not only the combinations of features as set out in the disclosed embodiments but also any other combination of features from different embodiments. Various features mentioned in the different embodiments can be combined with explicit mentioning of such combination or arrangement in an example embodiment or without any explicit mentioning. Furthermore, any of the embodiments and features described or depicted herein may be claimed in a separate claim and/or in any combination with any embodiment or feature described or depicted herein or with any of the features.

Some portions of this description describe the embodiments in terms of algorithms and symbolic representations of operations on information. These operations and algorithmic descriptions, while described functionally, computationally, or logically, are understood to be implemented by computer programs or equivalent electrical circuits, microcode, or the like. Furthermore, it has also proven convenient at times, to refer to these arrangements of operations as engines, without loss of generality. The described operations and their associated engines may be embodied in software, firmware, hardware, or any combinations thereof.

Any of the steps, operations, or processes described herein may be performed or implemented with one or more hardware or software engines, alone or in combination with other devices. In some embodiments, a software engine is implemented with a computer program product comprising a computer-readable medium containing computer program code, which can be executed by a computer processor for performing any or all of the steps, operations, or processes described. The term “steps” does not mandate or imply a particular order. For example, while this disclosure may describe a process that includes multiple steps sequentially with arrows present in a flowchart, the steps in the process do not need to be performed in the specific order claimed or described in the disclosure. Some steps may be performed before others even though the other steps are claimed or described first in this disclosure. Likewise, any use of (i), (ii), (iii), etc., or (a), (b), (c), etc. in the specification or in the claims, unless specified, is used to better enumerate items or steps and also does not mandate a particular order.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein. In addition, the term “each” used in the specification and claims does not imply that every or all elements in a group need to fit the description associated with the term “each.” For example, “each member is associated with element A” does not imply that all members are associated with an element A. Instead, the term “each” only implies that a member (of some of the members), in a singular form, is associated with an element A. In claims, the use of a singular form of a noun may imply at least one element even though a plural form is not used.

Finally, the language used in the specification has been principally selected for readability and instructional purposes, and it may not have been selected to delineate or circumscribe the patent rights. It is therefore intended that the scope of the patent rights be limited not by this detailed description, but rather by any claims that issue on an application based hereon. Accordingly, the disclosure of the embodiments is intended to be illustrative, but not limiting, of the scope of the patent rights.

The following applications are incorporated by reference in their entirety for all purposes: (1) U.S. Pat. No. 10,679,729, entitled “Haplotype Phasing Models,” granted on Jun. 9, 2020, (2) U.S. Pat. No. 10,223,498, entitled “Discovering Population Structure from Patterns of Identity-By-Descent,” granted on Mar. 5, 2019, (3) U.S. Pat. No. 10,720,229, entitled “Reducing Error in Predicted Genetic Relationships,” granted on Jul. 21, 2020, (4) U.S. Pat. No. 10,558,930, entitled “Local Genetic Ethnicity Determination System,” granted on Feb. 11, 2020, (5) U.S. Pat. No. 10,114,922, entitled “Identifying Ancestral Relationships Using a Continuous Stream of Input,” granted on Oct. 30, 2018, (6) U.S. Pat. No. 11,429,615, entitled “Linking Individual Datasets to a Database,” granted on Aug. 30, 2022, (7) U.S. Pat. No. 10,692,587, entitled “Global Ancestry Determination System,” granted on Jun. 23, 2020, and (8) U.S. Patent Application Publication No. US 2021/0034647, entitled “Clustering of Matched Segments to Determine Linkage of Dataset in a Database,” published on Feb. 4, 2021.

What is claimed is:

1. A computer-implemented method for predicting a relationship, comprising:
extracting a plurality of features between a first genetic dataset of a target individual and second genetic dataset of a match individual, the match individual being a genetic match of the target individual, wherein the plurality of features comprise: one or more genetic features shared between the first and second genetic datasets and an age difference between the target individual and the match individual; and
predicting, using a machine learning model and based on the extracted plurality of features, a number of generations between a most recent common ancestor (MRCA) and the target individual and a number of generations between the MRCA and the match individual.

2. The computer-implemented method of claim 1, wherein the match individual is identified by identity by descent (IBD) segments shared between the first genetic dataset and the second genetic dataset.

3. The computer-implemented method of claim 1, wherein the match individual is identified by centimorgans shared, a number of shared segments, or other genetic similarity with the target individual.

4. The computer-implemented method of claim 1, wherein the genetic features comprise: centimorgans shared and a number of shared segments between the two individuals.

5. The computer-implemented method of claim 1, wherein the predicted number of generations between the MRCA and the target individual and the number of generations between the MRCA and the match individual are used to generate a predicted relationship between the target individual and the match individual.

6. The computer-implemented method of claim 5, wherein the number of generations between the MRCA and the target individual and the number of generations between the MRCA and the match individual are used in combination with centimorgans shared between the target individual and the match individual, a number of shared DNA segments, and the age difference between the target individual and match individual to generate the predicted relationship between the target individual and the match individual.

7. The computer-implemented method of claim 1, wherein the machine learning model is trained on training samples, each training sample comprising an age difference between a pair of matched individuals, centimorgans between the pair, and a number of shared segments between the pair.

8. The computer-implemented method of claim 1, wherein training of the machine learning model comprises:
receiving training samples that comprise age differences between pairs of matched individuals and known generation data;
inputting the training samples to the machine learning model to generate predicted generations;
comparing the predicted generations to known generation data in the training samples; and
adjusting weights of the machine learning model based on the comparison.

9. A non-transitory computer-readable medium configured to store code comprising instructions for predicting a relationship, wherein the instructions, when executed by one or more processors, cause the one or more processors to perform steps comprising:
extracting a plurality of features between a first genetic dataset of a target individual and second genetic dataset of a match individual, the match individual being a genetic match of the target individual, wherein the plurality of features comprise: one or more genetic features shared between the first and second genetic datasets and an age difference between the target individual and the match individual; and
predicting, using a machine learning model and based on the extracted plurality of features, a number of generations between a most recent common ancestor (MRCA) and the target individual and a number of generations between the MRCA and the match individual.

10. The non-transitory computer-readable medium of claim 9, wherein the match individual is identified by identity by descent (IBD) segments shared between the first genetic dataset and the second genetic dataset.

11. The non-transitory computer-readable medium of claim 9, wherein the match individual is identified by centimorgans shared, a number of shared segments, or other genetic similarity with the target individual.

12. The non-transitory computer-readable medium of claim 9, wherein the genetic features comprise: centimorgans shared and a number of shared segments between the two individuals.

13. The non-transitory computer-readable medium of claim 9, wherein the predicted number of generations between the MRCA and the target individual and the number of generations between the MRCA and the match individual are used to generate a predicted relationship between the target individual and the match individual.

14. The non-transitory computer-readable medium of claim 9, wherein the number of generations between the MRCA and the target individual and the number of generations between the MRCA and the match individual are used in combination with centimorgans shared between the target individual and the match individual, a number of shared DNA segments, and the age difference between the target individual and match individual to generate the predicted relationship between the target individual and the match individual.

15. The non-transitory computer-readable medium of claim 9, wherein the machine learning model is trained on training samples, each training sample comprising an age difference between a pair of matched individuals, centimorgans between the pair, and a number of shared segments between the pair.

16. The non-transitory computer-readable medium of claim 9, wherein training of the machine learning model comprises:
receiving training samples that comprise age differences between pairs of matched individuals and known generation data;
inputting the training samples to the machine learning model to generate predicted generations;
comparing the predicted generations to known generation data in the training samples; and
adjusting weights of the machine learning model based on the comparison.

17. A system comprising one or more processors and one or more hardware storage devices having stored thereon computer-executable instructions that, when executed by the one or more processors, cause the one or more processors to:
extract a plurality of features between a first genetic dataset of a target individual and second genetic dataset of a match individual, the match individual being a genetic match of the target individual, wherein the plurality of features comprise: one or more genetic features shared between the first and second genetic datasets and an age difference between the target individual and the match individual; and
predict, using a machine learning model and based on the extracted plurality of features, a number of generations between a most recent common ancestor (MRCA) and the target individual and a number of generations between the MRCA and the match individual.

18. The system of claim 17, wherein the genetic features comprise: centimorgans shared and a number of shared segments between the two individuals.

19. The system of claim 17, wherein the predicted number of generations between the MRCA and the target individual and the number of generations between the MRCA and the match individual are used to generate a predicted relationship between the target individual and the match individual.

20. The system of claim 17, wherein the number of generations between the MRCA and the target individual and the number of generations between the MRCA and the match individual are used in combination with centimorgans shared between the target individual and the match individual, a number of shared DNA segments, and the age difference between the target individual and match individual to generate the predicted relationship between the target individual and the match individual.
